(19) United States
(12) Patent Application Publication
(10) Pub. No.: US 2023/0331694 A1
(43) Pub. Date: Oct. 19, 2023

(54) FAP-ACTIVATED COMPOUNDS FOR TREATMENT OF CANCER

(71) Applicant: The Johns Hopkins University, Baltimore, MD (US)

(72) Inventors: W. Nathaniel Brennen, Baltimore, MD (US); John T. Isaacs, Baltimore, MD (US); Samuel R. Denmeade, Baltimore, MD (US); Emmanuel S. Akinboye, Baltimore, MD (US)

(21) Appl. No.: 18/006,760

(22) PCT Filed: Jul. 29, 2021
(86) PCT No.: PCT/US2021/043731

§ 371 Date: Jan. 25, 2023

Related U.S. Application Data
(60) Provisional application No. 63/059,705, filed on Jul. 31, 2020.

(51) Int. Cl. A61K 45/06; C07D 401/12; C07D 471/04; A61P 35/04; C07D 401/14

(52) U.S. Cl. CPC C07D 401/12; A61K 45/06; A61P 35/04; C07D 401/14; C07D 471/04

(57) ABSTRACT

The present disclosure relates to FAP-activated prodrugs that can be used in the treatment of cancer, such as prostate cancer. The disclosure also relates to pharmaceutical compositions comprising the prodrugs, and related methods of treatment.

### CROSS-REFERENCE TO RELATED APPLICATIONS

This application claims the benefit of U.S. Provisional Pat. Application No. 63/059,705, filed on Jul. 31, 2020, which is incorporated by reference herein in its entirety.

### STATEMENT REGARDING FEDERALLY SPONSORED RESEARCH

This invention was made with government support under grant W81XWH-16-1-0410 awarded by the Department of Defense. The government has certain rights in the invention.

### FIELD

The present disclosure relates to FAP-activated prodrug compounds that can be used in the treatment of cancer, such as prostate cancer. The disclosure also relates to pharmaceutical compositions comprising the compounds, and related methods of treatment.

### BACKGROUND

Prostate cancer is the most commonly diagnosed cancer in men in the United States. It remains an incurable disease once progression to the metastatic castration-resistant (mCRPC) state occurs. Unfortunately, each of the FDA-approved agents for mCRPC produces only modest increases in overall survival followed by the emergence of resistance and a more aggressive phenotype.

### SUMMARY

Provided herein are compounds of formula (I):

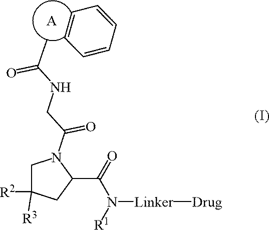

or a pharmaceutically acceptable salt thereof, wherein:
R¹ is selected from hydrogen and methyl;
R² and R³ are each independently halogen; and
A is a 5- or 6-membered heteroaryl or heterocyclic ring.

In some embodiments, R¹ is hydrogen. In some embodiments, R² and R³ are each fluoro. In some embodiments, A is a 5- or 6-membered heteroaryl having one heteroatom selected from N, O, and S. In some embodiments, A is selected from thiophene, furan, and pyridine.

In some embodiments, the compound has formula (Ia):

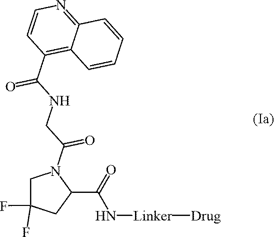

In some embodiments, the linker is a self-cleaving linker. In some embodiments, the linker has a formula selected from:

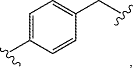

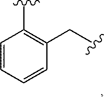

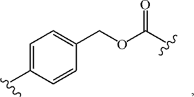

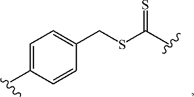

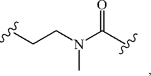

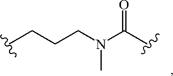

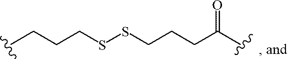

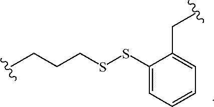

In some embodiments, the drug is selected from niclosamide, emetine, 2-hydroxyflutamide, and tasquinimod.

In some embodiments, the compound is selected from:

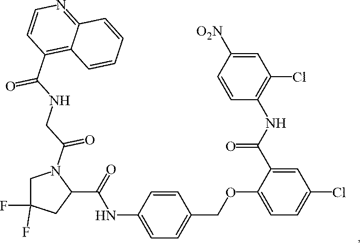

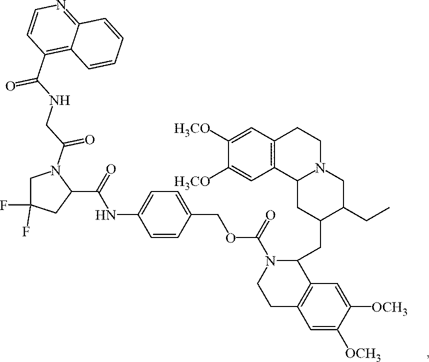

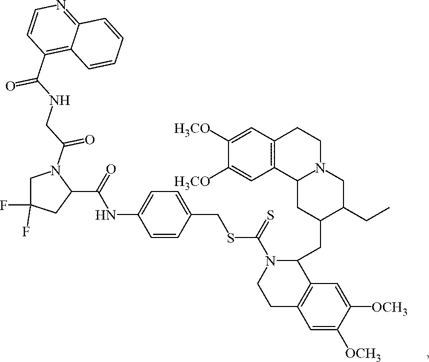

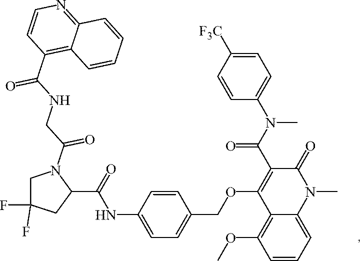

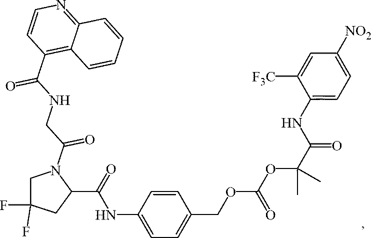

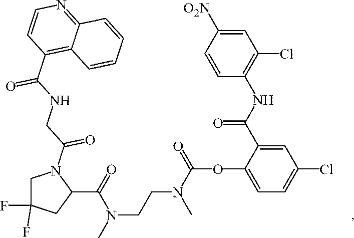

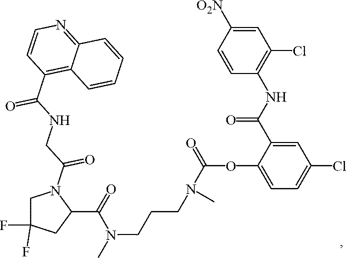

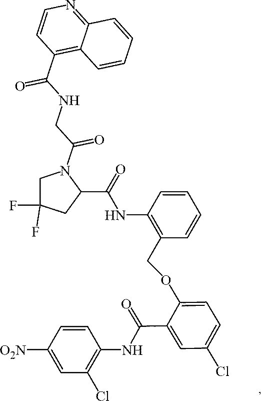

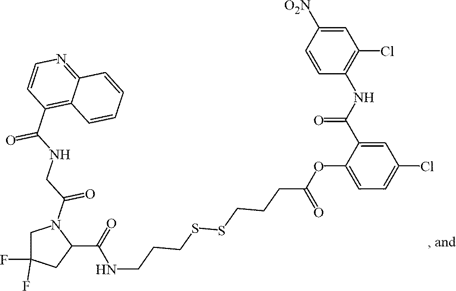

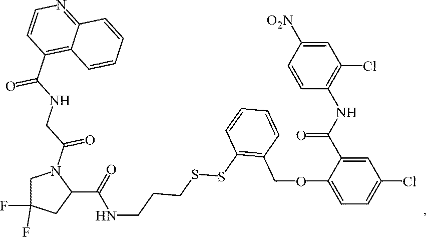

and a pharmaceutically acceptable salt thereof.

The disclosure also provides a pharmaceutical composition comprising a compound disclosed herein (i.e., a compound of formula (I)).

The disclosure also provides a method of treating cancer in a subject in need thereof, comprising administering to the subject a therapeutically effective amount of a compound disclosed herein (e.g., a compound of formula (I)). In some embodiments, the cancer is prostate cancer. In some embodiments, the prostate cancer is metastatic castration-resistant prostate cancer. In some embodiments, the method further comprises administering an additional chemotherapeutic agent to the subject. In some embodiments, the subject is a human.

The disclosure also provides a use of a compound disclosed herein (e.g., a compound of formula (I)) or a pharmaceutical composition disclosed herein (e.g., a pharmaceutical composition comprising a compound of formula (I)) in the treatment of cancer. In some embodiments, the cancer is prostate cancer. In some embodiments, the prostate cancer is metastatic castration-resistant prostate cancer.

### BRIEF DESCRIPTION OF THE DRAWINGS

FIG. 1 is a scheme showing how the drug niclosamide is released from a compound disclosed herein (Compound 1) following FAP cleavage.

FIGS. 2A-2C show data demonstrating that tumor-infiltrating FAP+ cells include mesenchymal stem cells (MSCs), cancer-associated fibroblasts (CAFs), and a subset of tumor-infiltrating CD11b+ myeloid cells. FIG. 2A shows results of a dual-label immunofluorescence assay documenting a significant fraction of FAP+ (green) cells are CD11b+ (red) myeloid cells in human mCRPC liver met. Nuclei stained with DAPI (blue). FIG. 2B shows results from a triple-labeled immunofluorescence assay for canonical MSC markers [CD73 (green), CD90 (pink), and CD105 (red)] identify human MSCs in archival FFPE human prostate cancer tissue. FIG. 2C shows flow cytometry data demonstrating that human prostate cancer-derived stromal cells (i.e. CAFs) express FAP, but not normal prostate stroma (nPrSCs).

FIGS. 3A-3D show characterization data for a compound disclosed herein (Compound 1). FIG. 3A shows data from a MTT assay documenting potency of niclosamide against a panel of benign and malignant cells. FIG. 3B shows LC-MS data demonstrating FAP-dependent production of active niclosamide from Compound 1. FIG. 3C shows data for Myc-CaP-CR tumors treated with abiraterone (Abi) (10 mg/kg) +/- FAP-activated Compound 1 (50 mg/kg). FIG. 3D shows data for the body weight (g) of treated animals.

FIG. 4 shows data demonstrating that Compound 4 decreases tumor growth compared to a control.

FIG. 5 shows in vitro kinetic data for FAP-dependent activation of Compound 1 in the presence and absence of BSA.

FIG. 6 shows data from a mitochondrial stress test assay of Compound 1, showing the oxygen consumption rate (OCR).

FIG. 7 shows data from a mitochondrial stress test assay of Compound 1, showing the extracellular acidification rate (ECAR).

FIG. 8 shows tumor volume and body weight data for castrated male NSG mice inoculated with castration-resistant CWR22-H tumors and treated with Compound 1.

FIGS. 9A-9D show pharmacokinetic data in plasma (FIG. 9A), liver (FIG. 9B), kidney (FIG. 9C), and smooth muscle (FIG. 9D) following dosing with Compound 1.

FIG. 10 shows data demonstrating FAP-dependent activation of Compound 4 in vitro.

FIG. 11 shows overexpression of FAP in primary and metastatic human prostate cancer via immunohistochemical staining compared to adjacent benign prostate tissue.

### DETAILED DESCRIPTION

Provided herein are compounds in which a cancer drug molecule is chemically coupled to a small, water-soluble dipeptide substrate for fibroblast activation protein (FAP), which is a membrane-bound extracellular serine protease belonging to the S9B prolyl oligopeptidase subfamily. FAP is not typically expressed in normal tissues but is upregulated in prostate cancer (see, e.g., Kesch et al. Eur. J. Nucl. Med. Mol. Imaging (2021) doi: 10.1007/s00259-021-05423-y; Brennen et al. Immunotherapy 12, 155-175 (2021); Hintz et al. Clin. Cancer Res. 26, 4882-4891 (2020); Brennen et al. J. Natl. Cancer Inst. 104, 1320-1334 (2012)). FAP is highly upregulated in the stroma of >90% of all solid tumors, and infiltration of FAP-positive cells (e.g., tumor-associated macrophages (TAMs), mesenchymal stem cells (MSCs), and carcinoma-associated fibroblasts (CAFs)) is a key feature of primary and metastatic human prostate cancer (see, e.g., Krueger et al. Prostate 79, 320-330, (2019); Brennen et al. Oncotarget 4, 106-117 (2013); Brennen et al. Prostate 76, 552-564 (2016); Brennen et al. Oncotarget 7, 71298-71308 (2016); Brennen et al. Oncotarget 8, 46710-46727 (2017); Krueger et al. Stem Cells Transl Med, doi:10.1002/sctm.18-0024 (2018). Linking a drug molecule to an FAP substrate allows for selective release of the drug in environments in which FAP is present and enzymatically active, such as the tumor microenvironment.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents, and other references mentioned herein are incorporated by reference in their entirety. The materials, methods, and examples disclosed herein are illustrative only and not intended to be limiting.

For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Sorrell, Organic Chemistry, 2^{nd} edition, University Science Books, Sausalito, 2006; Smith, March’s Advanced Organic Chemistry: Reactions, Mechanism, and Structure, 7^{th} Edition, John Wiley & Sons, Inc., New York, 2013; Larock, Comprehensive Organic Transformations, 3^{rd} Edition, John Wiley & Sons, Inc., New York, 2018; and Carruthers, Some Modern Methods of Organic Synthesis, 3^{rd} Edition, Cambridge University Press, Cambridge, 1987; the entire contents of each of which are incorporated herein by reference.

As used herein, the term “halogen” or “halo” means F, Cl, Br, or I.

As used herein, the term “heteroaryl” refers to an aromatic group having a single ring (monocyclic) or multiple rings (bicyclic or tricyclic), having one or more ring heteroatoms independently selected from O, N, and S. The aromatic monocyclic rings are five- or six-membered rings containing at least one heteroatom independently selected from O, N, and S (e.g. 1, 2, 3, or 4 heteroatoms independently selected from O, N, and S). The five-membered aromatic monocyclic rings have two double bonds, and the six- membered aromatic monocyclic rings have three double bonds. The bicyclic heteroaryl groups are exemplified by a monocyclic heteroaryl ring appended fused to a monocyclic aryl group, as defined herein, or a monocyclic heteroaryl group, as defined herein. The tricyclic heteroaryl groups are exemplified by a monocyclic heteroaryl ring fused to two rings independently selected from a monocyclic aryl group, as defined herein, and a monocyclic heteroaryl group as defined herein. Representative examples of monocyclic heteroaryl include, but are not limited to, pyridinyl (including pyridin-2-yl, pyridin-3-yl, pyridin-4-yl), pyrimidinyl, pyrazinyl, pyridazinyl, pyrrolyl, benzopyrazolyl, 1,2,3-triazolyl, 1,3,4-thiadiazolyl, 1,2,4-thiadiazolyl, 1,3,4-oxadiazolyl, 1,2,4-oxadiazolyl, imidazolyl, thiazolyl, isothiazolyl, thienyl, furanyl, oxazolyl, isoxazolyl, 1,2,4-triazinyl, and 1,3,5-triazinyl. Representative examples of bicyclic heteroaryl include, but are not limited to, benzimidazolyl, benzodioxolyl, benzofuranyl, benzooxadiazolyl, benzopyrazolyl, benzothiazolyl, benzothienyl, benzotriazolyl, benzoxadiazolyl, benzoxazolyl, chromenyl, imidazopyridine, imidazothiazolyl, indazolyl, indolyl, isobenzofuranyl, isoindolyl, isoquinolinyl, naphthyridinyl, purinyl, pyridoimidazolyl, quinazolinyl, quinolinyl, quinoxalinyl, thiazolopyridinyl, thiazolopyrimidinyl, thienopyrrolyl, and thienothienyl. Representative examples of tricyclic heteroaryl include, but are not limited to, dibenzofuranyl and dibenzothienyl. The monocyclic, bicyclic, and tricyclic heteroaryls are connected to the parent molecular moiety through any carbon atom or any nitrogen atom contained within the rings.

As used herein, the term “heterocycle” or “heterocyclic” refers to a saturated or partially unsaturated non-aromatic cyclic group having one or more ring heteroatoms independently selected from O, N, and S. means a monocyclic heterocycle, a bicyclic heterocycle, or a tricyclic heterocycle. The monocyclic heterocycle is a three-, four-, five-, six-, seven-, or eight-membered ring containing at least one heteroatom independently selected from O, N, and S. The three- or four-membered ring contains zero or one double bond, and one heteroatom selected from O, N, and S. The five-membered ring contains zero or one double bond and one, two or three heteroatoms selected from O, N and S. The six-membered ring contains zero, one, or two double bonds and one, two, or three heteroatoms selected from O, N, and S. The seven- and eight-membered rings contains zero, one, two, or three double bonds and one, two, or three heteroatoms selected from O, N, and S. Representative examples of monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, oxetanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a phenyl group, or a monocyclic heterocycle fused to a monocyclic cycloalkyl, or a monocyclic heterocycle fused to a monocyclic cycloalkenyl, or a monocyclic heterocycle fused to a monocyclic heterocycle, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. Representative examples of bicyclic heterocycles include, but are not limited to, benzopyranyl, benzothiopyranyl, chromanyl, 2,3-dihydrobenzofuranyl, 2,3-dihydrobenzothienyl, 2,3-dihydroisoquinoline, 2-azaspiro[3.3]heptan-2-yl, azabicyclo[2.2.1 ]heptyl (including 2-azabicyclo[2.2.1]hept-2-yl), 2,3-dihydro-1H-indolyl, isoindolinyl, octahydrocyclopenta[c]pyrrolyl, octahydropyrrolopyridinyl, and tetrahydroisoquinolinyl. Tricyclic heterocycles are exemplified by a bicyclic heterocycle fused to a phenyl group, or a bicyclic heterocycle fused to a monocyclic cycloalkyl, or a bicyclic heterocycle fused to a monocyclic cycloalkenyl, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan,hexahydro-1H-1,4-methanocyclopenta[c]furan, aza-adamantane (1-azatricyclo[3.3.1.1^{3,7}]decane), and oxa-adamantane (2-oxatricyclo[3.3.1.1^{3,7}]decane). The monocyclic, bicyclic, and tricyclic heterocycles are connected to the parent molecular moiety through any carbon atom or any nitrogen atom contained within the rings.

As used herein, the term “subject” broadly refers to any animal, including but not limited to, human and non-human animals (e.g., dogs, cats, cows, horses, sheep, poultry, etc.). As used herein, the term “patient” typically refers to a subject that is being treated for a disease or condition.

As used herein, the terms “treatment,” “treat,” and “treating” refer to reversing, alleviating, delaying the onset of, or inhibiting the progress of a disease or condition, or one or more signs or symptoms thereof. In some embodiments, “treatment,” “treat,” and “treating” require that signs or symptoms of the disease disorder or condition have developed or have been observed. In other embodiments, treatment may be administered in the absence of signs or symptoms of the disease or condition. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “substantially” means that the recited characteristic, parameter, and/or value need not be achieved exactly, but that deviations or variations, including for example, tolerances, measurement error, measurement accuracy limitations, and other factors known to skill in the art, may occur in amounts that do not preclude the effect the characteristic was intended to provide. A characteristic or feature that is substantially absent may be one that is within the noise, beneath background, below the detection capabilities of the assay being used, or a small fraction (e.g., <1%, <0.1%, <0.01%, <0.001%, <0.00001%, <0.000001%, <0.0000001%) of the significant characteristic.

### 2. Compounds

The present disclosure includes compounds that are prodrugs of anti-cancer compounds, particularly anti-cancer compounds that are useful in treating solid tumors such as prostate cancer in which FAP is upregulated. The compounds include an FAP substrate moiety, a linker, and a drug. The compounds of formula (I) have formula:

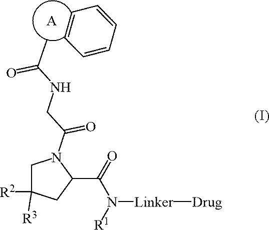

wherein R¹ is selected from hydrogen and methyl, R² and R³ are each independently halogen, and A is a 5- or 6-membered heteroaryl or heterocyclic ring.

In some embodiments, the compound of formula (I) has formula (Ia):

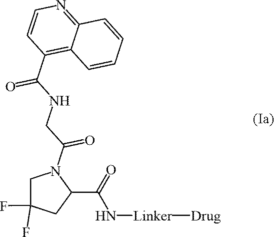

### A. FAP Substrate Moiety

The compounds include a small, orally active, water-soluble dipeptide substrate for FAP, having nanomolar affinity and >80 fold specificity over all closely-related dipeptidyl peptidase family members, including prolyl oligopeptidase (PREP) and dipeptidyl peptidase IV (DPPIV). FAP-dependent cleavage of the compound via proteolysis leads to cleavage of the linker and release of the drug. Because high levels of FAP expression are restricted to cells found within the tumor microenvironment, this approach spares toxicity to normal tissues while maintaining anti-tumor efficacy once activated. The FAP substrate moiety has the following formula:

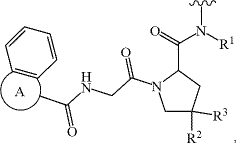

wherein R¹ is selected from hydrogen and methyl, R² and R³ are each independently halogen, and A is a 5- or 6-membered heteroaryl or heterocyclic ring. In some embodiments, R¹ is hydrogen. In some embodiments, R¹ is methyl. In some embodiments, R² and R³ are each independently selected from fluoro and chloro. In some embodiments, R² and R³ are each fluoro. In some embodiments, A is a 5- or 6-membered heteroaryl having one heteroatom selected from N, O, and S. In some embodiments, A is selected from thiophene, furan, and pyridine. In some embodiments, A is pyridine. In some embodiments, A is a 5- or 6-membered heterocyclic ring having one heteroatom selected from N, O, and S. In some embodiments, A is pyran or thiopyran.

In some embodiments, the FAP substrate moiety has the following formula:

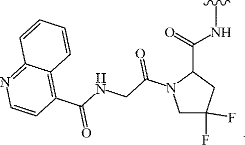

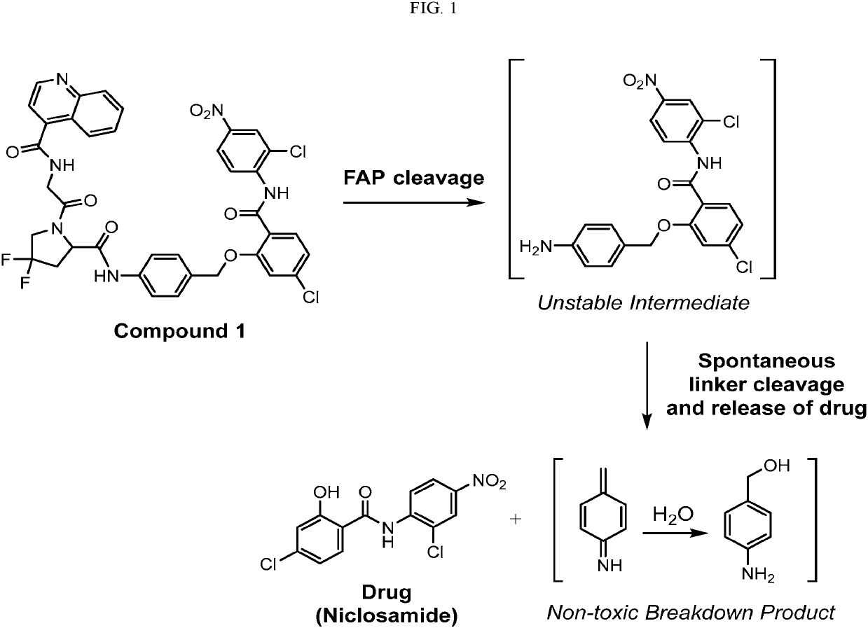

When the compound is in an environment in which active FAP is present (e.g., a tumor microenvironment), FAP cleaves the compound to release the Linker-Drug molecule, which is then further processed as discussed below, and as generally illustrated for an exemplary compound in FIG. 1.

### B. Linkers

The compounds include a linker that links the FAP substrate moiety and the drug. Although a variety of linkers can be used, in particular embodiments, the linker is a self-cleaving linker. Such a linker can cleave spontaneously after FAP-dependent cleavage of the FAP moiety, releasing the drug compound at the site of interest.

The linker can include one or more groups independently selected from methylene (—CH₂—), ether (—O—), amine (—NH—), alkylamine (—NR—, wherein R is an optionally substituted C₁-C₆ alkyl group), thioether (—S—), disulfide (—S—S—), amide (—C(O)NH—), ester (—C(O)O—), carbamate (—OC(O)NH—), sulfonamide (—S(O)₂NH—), phenylene (—C₆H₄—), and any combination thereof. Exemplary linkers include the following:

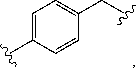

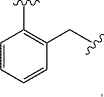

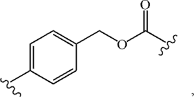

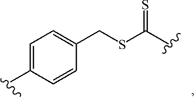

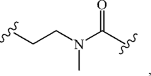

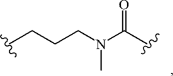

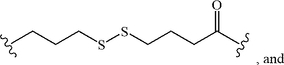

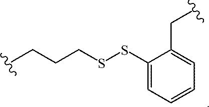

An exemplary self-cleavage reaction with an exemplary linker is shown in FIG. 1. Other linkers exemplified above will cleave in a similar manner (e.g., with concomitant release of CO₂ or CS₂). Certain linkers will cyclize to liberate a 5- or 6-membered rink along with the active drug compound. For linkers with disulfide groups, the compounds will first be activated by FAP in the tumor microenvironment. Then, the remaining drug-linker conjugate will undergo endocytosis, and intracellular sulfhydryl groups (e.g., cysteine, glutathione, or the like) will cleave the disulfide bond. After such cleavage, spontaneous release of the drug compound will occur intracellularly either by cyclization or by rearrangement.

### C. Drugs

The compounds include a drug molecule that includes a functional group, such as an amino or hydroxy group, that is important for the drug molecule’s biological activity. In particular embodiments, the drug molecule is an anti-cancer agent. Particular anti-cancer agents include those useful for treating cancer, including compounds for treating solid tumors such as prostate cancer. In some embodiments, the anti-cancer agent has a functional group, such as a hydroxy group or an amine, that is important for the function of the compound. Attachment of the linker at this position can prevent the drug from exerting any activity until it is released following proteolysis of the FAP substrate moiety and cleavage of the linker.

In some embodiments, the drug is selected from niclosamide, emetine, 2-hydroxyflutamide, and tasquinimod.

### I. Niclosamide

Niclosamide is an FDA-approved oral salicylanilide anti-helminthic used to treat tapeworms based on its ability to uncouple oxidative phosphorylation and stimulate ATPase activity in the mitochondria of the parasites, resulting in their death (Li et al. Cancer Lett 349, 8-14 (2014)). Due to this uncoupling ability, niclosamide is essentially lethal to all cells with an LC₅₀ <1 µM, including mammalian cells such as mCRPCs, TAMs, MSCs, and CAFs (Liu et al. Clin Cancer Res 20, 3198-3210 (2014); Jin et al. Cancer Res 70, 2516-2527 (2010)). This cytotoxicity is due to its high lipophilicity and thus high passive cell penetration, where it enters mitochondria, collapses the proton gradient needed for ATP synthesis, and increases production of ROS (Park et al. BMB Rep 44, 517-522 (2011); Fonseca et al. J Biol Chem 287, 17530-17545 (2012); Terada et al. Environ Health Perspect 87, 213-218 (1990).

Niclosamide (pKa of 5.6) is ionized at neutral pH, and the anion readily passes through biological membranes, including the mitochondrial outer membrane, due to its lipophilic nature (log P = 4.48 at pH 7.0). Once in the acidic intramembranous mitochondrial space (IMS), anionic niclosamide is protonated, increasing its lipophilicity (log P = 5.63 at pH 5.7) and enhancing its translocation across the outer mitochondrial membrane into the pH-neutral cytosol where a proton dissociates, which allows the anionic compound to translocate back across the outer mitochondrial membrane into the IMS to repeat the cycle (Fonseca 2012, Terada 1990), and thus collapse the mitochondrial proton gradient.

Niclosamide is safe and effective against gut tapeworms due to its limited water solubility and absorption. However, this poor oral bioavailability limits its effectiveness as a systemic anti-cancer agent. Herein, to overcome its low therapeutic index and increase its solubility/bioavailability, niclosamide has been coupled to the FAP substrate via its phenolic hydroxyl group, which is essential to niclosamide’s mechanism of action, such that the compound is only active in the presence of enzymatically active FAP.

Accordingly, in some embodiments, in compounds of formula (I), the drug has formula:

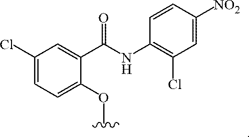

### II. Emetine

Emetine is a natural product alkaloid found in the root of Psychotria ipecacuanha. It is the active ingredient in the ipecac root used in traditional folk medicine as an emetic and expectorant. It is used to induce vomiting in the event of accidental ingestion of toxic agents, and subcutaneous injection of emetine has also been used to treat amoebiasis, amebic dysentery, and trypanosomiasis. Its primary mechanism of action appears to be related to its ability to inhibit ribosomal and mitochondrial protein synthesis (Grollman, Proc. Natl. Acad. Sci. USA 1966, 56(6), 1867-1874; Lietman, Mol. Pharmacol. 1970, 7:122-128). Its anticancer activities were investigated in several phase I-II clinical trials in a number of solid tumors in the early 1970s and although clinical responses were observed, emetine was reported to have a very narrow therapeutic index, and dose-dependent side effects such as muscle fatigue and cardiac toxicity were observed. (Panettiere et al. Cancer 1971, 27, 835-841; Kane et al. Cancer Chemother. Rep. 1975, 59, 1171-1172; Siddiqui et al. Cancer Chemother. Rep. 1973, 57, 423-428; Moertel et al. Cancer Chemother. Rep. 1974, 58, 229-232; Street, Lancet 1972, 2, 281-282; Mastrangelo et al. Cancer 1973, 31, 1170-1175). Structure-activity relationship studies have demonstrated a relatively significant loss of protein synthesis inhibitory activity in N-methylemetine (see, e.g., Grollman, Proc. Natl. Acad. Sci. USA 1966, 56(6), 1867-1874). Accordingly, linkage of emetine to the FAP substrate via its secondary amino group can ensure that the compound will only be active upon reaching the tumor microenvironment while remaining relatively inactive in general circulation and in normal tissues.

Accordingly, in some embodiments, in compounds of formula (I), the drug has formula:

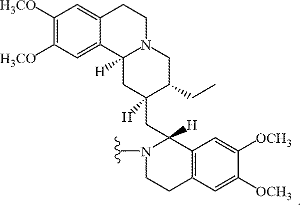

### Iii. Tasquinimod

Tasquinimod is a compound currently being investigated for treatment of solid tumors, including prostate cancer. It targets the tumor microenvironment and counteracts cancer development by inhibiting angiogenesis and metastasis and by modulating the immune system (Isaacs et al. Prostate 2006, 66(16), 1768-1778; Isaacs et al. Cancer Res. 2012, 73(4), 1386-1399; Kallberg et al. PLoS ONE 2012, 7(3), e34207; Jennbacken et al. Prostate 2012, 72(8), 913-924).

Accordingly, in some embodiments, in compounds of formula (I), the drug has formula:

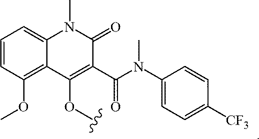

### IV. 2-Hydroxyflutamide

2-hydroxyflutamide is a non-steroidal antiandrogen, and is the major active metabolite of flutamide, which is used to treat prostate cancer along with other androgen-dependent conditions. Flutamide is considered to be a prodrug of 2-hydroxyflutamide. 2-hydroxyflutamide has a relatively short half-life, and is considerably less potent than other prostate cancer drugs. Linkage of the 2-hydroxy group to the FAP substrate can ensure that the compound will only be active upon reaching the tumor microenvironment while remaining relatively inactive in general circulation and in normal tissues, which can result in increased potency at the site of interest.

Accordingly, in some embodiments, in compounds of formula (I), the drug has formula:

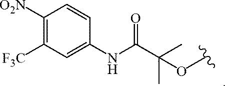

### D. Exemplary Compounds

In some embodiments, the compound of formula (I) is selected from:

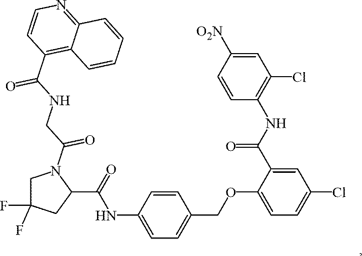

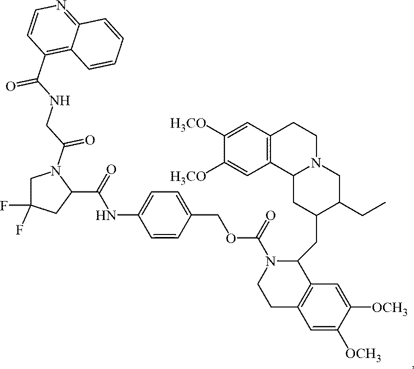

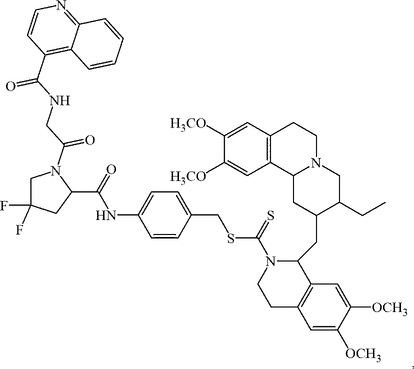

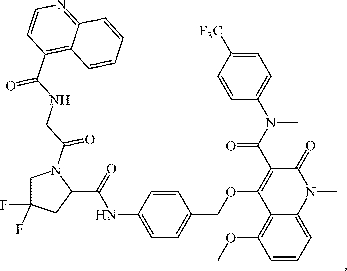

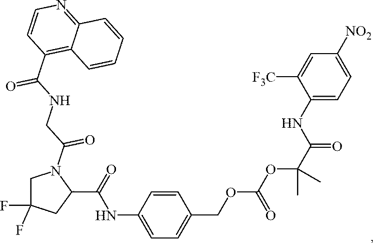

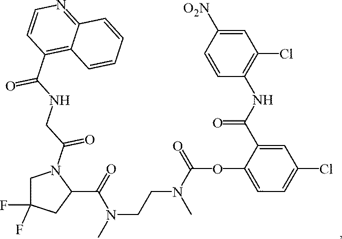

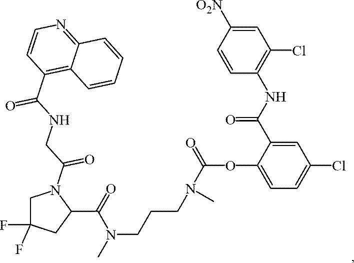

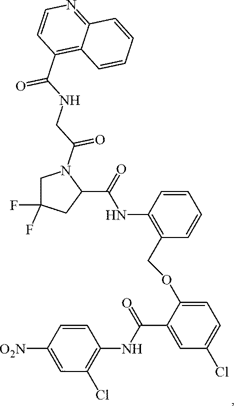

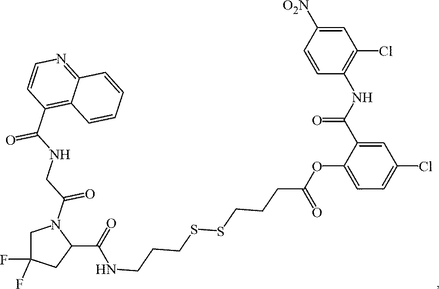

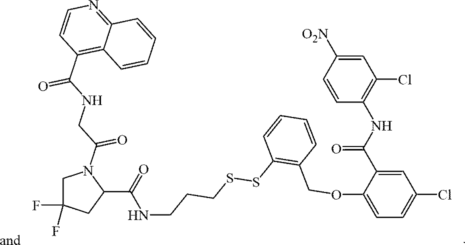

### E. Salt Forms and Isomers

The compounds can be in the form of a salt. In some embodiments, a neutral form of the compound may be regenerated by contacting the salt with a base or acid and isolating the parent compound in a conventional manner. The parent form of the compound differs from the various salt forms in certain physical properties, such as solubility in polar solvents, but otherwise the salts are equivalent to the parent form of the compound for the purposes of this disclosure.

In particular, if the compound is anionic or has a functional group that may be anionic (e.g., —COOH may be —COO⁻, —SO₃H may be —SO₃¯, or —P(O)(OH)₂ can be —PO₃²⁻), then a salt may be formed with one or more suitable cations. Examples of suitable inorganic cations include, but are not limited to, alkali metal cations such as Li⁺, Na⁺, and K⁺, alkaline earth cations such as Ca²⁺ and Mg²⁺, and other cations. Sodium salts may be particularly suitable. Examples of suitable organic cations include, but are not limited to, ammonium ion (i.e., NH₄⁺) and substituted ammonium ions (e.g., NH₃R₁⁺, NH₂R₂⁺, NHR₃⁺, and NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids such as lysine and arginine. In some embodiments, the compound is a sodium salt.

If the compound is cationic or has a functional group that may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous. Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucoheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, tetrafluoroboric, toluenesulfonic, trifluoromethanesulfonic, and valeric. In some embodiments, the compound is a halide salt, such as a chloro, bromo, or iodo salt. In some embodiments, the compound is a tetrafluoroborate or trifluoromethanesulfonate salt.

### F. Methods of Synthesis

The compounds can be prepared by a variety of methods. For example, compounds can be prepared as illustrated in Scheme 1. This synthesis is particularly suitable for drug molecules having a hydroxy group, via which the drug molecule is linked to the remainder of the compound. Abbreviations used in Scheme 1 include the following: DCM is dichloromethane; DIAD is diisopropyl azodicarboxylate; DMF is N,N-dimethylformamide; EEDQ is N-ethoxycarbonyl-2-ethoxy-1,2-dihydroquinoline; and THF is tetrahydrofuran.

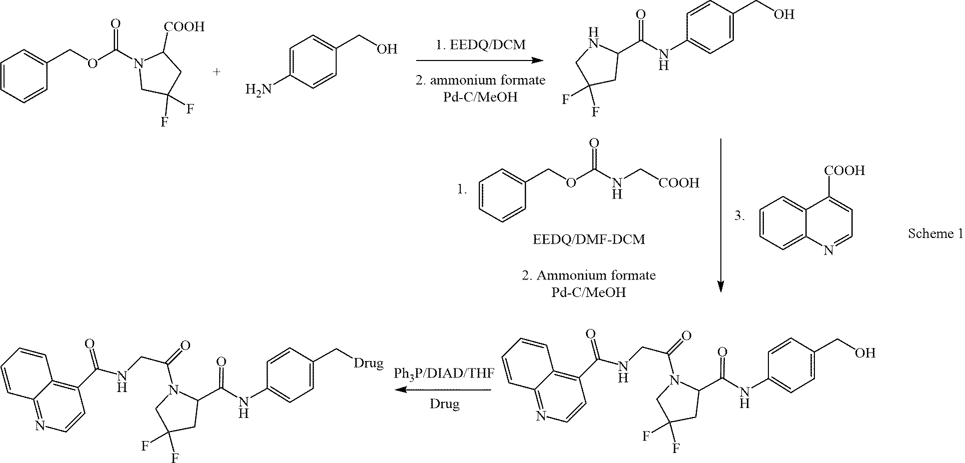

The compounds and intermediates herein may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration as described for instance in “Vogel’s Textbook of Practical Organic Chemistry,” 5th edition (1989), by Furniss, Hannaford, Smith, and Tatchell, pub. Longman Scientific & Technical, Essex CM20 2JE, England.

Reaction conditions and reaction times for each individual step can vary depending on the particular reactants employed and substituents present in the reactants used. Specific procedures are provided in the Examples section. Reactions can be worked up in the conventional manner, e.g., by eliminating the solvent from the residue and further purified according to methodologies generally known in the art such as, but not limited to, crystallization, distillation, extraction, trituration, and chromatography. Unless otherwise described, the starting materials and reagents are either commercially available or can be prepared by one skilled in the art from commercially available materials using methods described in the chemical literature. Starting materials, if not commercially available, can be prepared by procedures selected from standard organic chemical techniques, techniques that are analogous to the synthesis of known, structurally similar compounds, or techniques that are analogous to the above described schemes or the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the invention. Suitable protecting groups, and the methods for protecting and deprotecting different substituents using such suitable protecting groups, are well known to those skilled in the art; examples of which can be found in the treatise by PGM Wuts entitled “Greene’s Protective Groups in Organic Synthesis” (5th ed.), John Wiley & Sons, Inc. (2014), which is incorporated herein by reference in its entirety. Synthesis of the compounds of the invention can be accomplished by methods analogous to those described in the synthetic schemes described hereinabove and in specific examples.

When an optically active form of a disclosed compound is required, it can be obtained by carrying out one of the procedures described herein using an optically active starting material (prepared, for example, by asymmetric induction of a suitable reaction step) or by resolution of a mixture of the stereoisomers of the compound or intermediates using a standard procedure (such as chromatographic separation, recrystallization or enzymatic resolution).

Similarly, when a pure geometric isomer of a compound is required, it can be obtained by carrying out one of the above procedures using a pure geometric isomer as a starting material or by resolution of a mixture of the geometric isomers of the compound or intermediates using a standard procedure such as chromatographic separation.

The synthetic schemes and specific examples as described are illustrative and are not to be read as limiting the scope of the invention as it is defined in the claims. All alternatives, modifications, and equivalents of the synthetic methods and specific examples are included within the scope of the claims.

### 3. Pharmaceutical Compositions

The disclosed compounds may be incorporated into pharmaceutical compositions suitable for administration to a subject (such as a patient, which may be a human or non-human). Accordingly, in some embodiments, the present disclosure provides a pharmaceutical composition comprising a compound disclosed herein (i.e. a compound of formula (I), or a pharmaceutically acceptable salt thereof, and a pharmaceutically acceptable carrier.

The pharmaceutical compositions may include a “therapeutically effective amount” or a “prophylactically effective amount” of the agent. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the disclosure (e.g., a compound of formula (I)) are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

For example, a therapeutically effective amount of a compound of formula (I), may be about 1 mg/kg to about 1000 mg/kg, about 5 mg/kg to about 950 mg/kg, about 10 mg/kg to about 900 mg/kg, about 15 mg/kg to about 850 mg/kg, about 20 mg/kg to about 800 mg/kg, about 25 mg/kg to about 750 mg/kg, about 30 mg/kg to about 700 mg/kg, about 35 mg/kg to about 650 mg/kg, about 40 mg/kg to about 600 mg/kg, about 45 mg/kg to about 550 mg/kg, about 50 mg/kg to about 500 mg/kg, about 55 mg/kg to about 450 mg/kg, about 60 mg/kg to about 400 mg/kg, about 65 mg/kg to about 350 mg/kg, about 70 mg/kg to about 300 mg/kg, about 75 mg/kg to about 250 mg/kg, about 80 mg/kg to about 200 mg/kg, about 85 mg/kg to about 150 mg/kg, and about 90 mg/kg to about 100 mg/kg.

The pharmaceutical compositions include pharmaceutically acceptable carriers. The term “pharmaceutically acceptable carrier,” as used herein, means a nontoxic, inert solid, semisolid or liquid filler, diluent, encapsulating material auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer’s solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Thus, the compounds and their physiologically acceptable salts may be formulated for administration by, for example, solid dosing, eye drop, in a topical oil-based formulation, injection, inhalation (either through the mouth or the nose), implants, or oral, buccal, parenteral, or rectal administration. Techniques and formulations may generally be found in “Remington’s Pharmaceutical Sciences” (Meade Publishing Co., Easton, Pa.). Therapeutic compositions must typically be sterile and stable under the conditions of manufacture and storage.

The route by which the disclosed compounds are administered and the form of the composition will dictate the type of carrier to be used. The composition may be in a variety of forms, suitable, for example, for systemic administration (e.g., oral, rectal, nasal, sublingual, buccal, implants, or parenteral) or topical administration (e.g., dermal, pulmonary, nasal, aural, ocular, liposome delivery systems, or iontophoresis).

Carriers for systemic administration typically include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90%.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition is typically about 5 to about 10%.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50%.

Suitable disintegrants include agar, alginic acid and the sodium salt thereof, effervescent mixtures, croscarmellose, crospovidone, sodium carboxymethyl starch, sodium starch glycolate, clays, and ion exchange resins. The amount of disintegrant(s) in a systemic or topical composition is typically about 0.1 to about 10%.

Suitable colorants include a colorant such as an FD&C dye. When used, the amount of colorant in a systemic or topical composition is typically about 0.005 to about 0.1%.

Suitable flavors include menthol, peppermint, and fruit flavors. The amount of flavor(s), when used, in a systemic or topical composition is typically about 0.1 to about 1.0%.

Suitable sweeteners include aspartame and saccharin. The amount of sweetener(s) in a systemic or topical composition is typically about 0.001 to about 1%.

Suitable antioxidants include butylated hydroxyanisole (“BHA”), butylated hydroxytoluene (“BHT”), and vitamin E. The amount of antioxidant(s) in a systemic or topical composition is typically about 0.1 to about 5%.

Suitable preservatives include benzalkonium chloride, methyl paraben and sodium benzoate. The amount of preservative(s) in a systemic or topical composition is typically about 0.01 to about 5%.

Suitable glidants include silicon dioxide. The amount of glidant(s) in a systemic or topical composition is typically about 1 to about 5%.

Suitable solvents include water, isotonic saline, ethyl oleate, glycerine, hydroxylated castor oils, alcohols such as ethanol, and phosphate buffer solutions. The amount of solvent(s) in a systemic or topical composition is typically from about 0 to about 100%.

Suitable suspending agents include AVICEL RC-591 (from FMC Corporation of Philadelphia, PA) and sodium alginate. The amount of suspending agent(s) in a systemic or topical composition is typically about 1 to about 8%.

Suitable surfactants include lecithin, Polysorbate 80, and sodium lauryl sulfate, and the TWEENS from Atlas Powder Company of Wilmington, Delaware. Suitable surfactants include those disclosed in the C.T.F.A. Cosmetic Ingredient Handbook, 1992, pp.587-592; Remington’s Pharmaceutical Sciences, 15th Ed. 1975, pp. 335-337; and McCutcheon’s Volume 1, Emulsifiers & Detergents, 1994, North American Edition, pp. 236-239. The amount of surfactant(s) in the systemic or topical composition is typically about 0.1% to about 5%.

Although the amounts of components in the systemic compositions may vary depending on the type of systemic composition prepared, in general, systemic compositions include 0.01% to 50% of an active compound (e.g., a compound of formula (I)) and 50% to 99.99% of one or more carriers. Compositions for parenteral administration typically include 0.1% to 10% of actives and 90% to 99.9% of a carrier including a diluent and a solvent.

Compositions for oral administration can have various dosage forms. For example, solid forms include tablets, capsules, granules, and bulk powders. These oral dosage forms include a safe and effective amount, usually at least about 5%, and more particularly from about 25% to about 50% of actives. The oral dosage compositions include about 50% to about 95% of carriers, and more particularly, from about 50% to about 75%.

Tablets can be compressed, tablet triturates, enteric-coated, sugar-coated, film-coated, or multiple-compressed. Tablets typically include an active component, and a carrier comprising ingredients selected from diluents, lubricants, binders, disintegrants, colorants, flavors, sweeteners, glidants, and combinations thereof. Specific diluents include calcium carbonate, sodium carbonate, mannitol, lactose and cellulose. Specific binders include starch, gelatin, and sucrose. Specific disintegrants include alginic acid and croscarmellose. Specific lubricants include magnesium stearate, stearic acid, and talc. Specific colorants are the FD&C dyes, which can be added for appearance. Chewable tablets preferably contain sweeteners such as aspartame and saccharin, or flavors such as menthol, peppermint, fruit flavors, or a combination thereof.

Capsules (including implants, time release and sustained release formulations) typically include an active compound (e.g., a compound of formula (I)), and a carrier including one or more diluents disclosed above in a capsule comprising gelatin. Granules typically comprise a disclosed compound, and preferably glidants such as silicon dioxide to improve flow characteristics. Implants can be of the biodegradable or the non-biodegradable type.

The selection of ingredients in the carrier for oral compositions depends on secondary considerations like taste, cost, and shelf stability, which are not critical for the purposes of this disclosure.

Solid compositions may be coated by conventional methods, typically with pH or time-dependent coatings, such that a disclosed compound is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methyl cellulose phthalate, ethyl cellulose, EUDRAGIT® coatings (available from Evonik Industries of Essen, Germany), waxes and shellac.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include a disclosed compound and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

Other compositions useful for attaining systemic delivery of the subject compounds include sublingual, buccal and nasal dosage forms. Such compositions typically include one or more of soluble filler substances such as diluents including sucrose, sorbitol and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

The disclosed compounds can be topically administered. Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. Topical compositions include: a disclosed compound (e.g., a compound of formula (I)), and a carrier. The carrier of the topical composition preferably aids penetration of the compounds into the skin. The carrier may further include one or more optional components.

The amount of the carrier employed in conjunction with a disclosed compound is sufficient to provide a practical quantity of composition for administration per unit dose of the compound. Techniques and compositions for making dosage forms useful in the methods of this disclosure are described in the following references: Modern Pharmaceutics, Chapters 9 and 10, Banker & Rhodes, eds. (1979); Lieberman et al, Pharmaceutical Dosage Forms: Tablets (1981); and Ansel, Introduction to Pharmaceutical Dosage Forms, 2nd Ed., (1976).

A carrier may include a single ingredient or a combination of two or more ingredients. In the topical compositions, the carrier includes a topical carrier. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols.

The carrier of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane- 1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5% to about 95%.

Suitable propellants include propane, butane, isobutane, dimethyl ether, carbon dioxide, nitrous oxide, and combinations thereof. The amount of propellant(s) in a topical composition is typically about 0% to about 95%.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95%.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95%.

The amount of thickener(s) in a topical composition is typically about 0% to about 95%.

Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95%.

The amount of fragrance in a topical composition is typically about 0% to about 0.5%, particularly, about 0.001% to about 0.1%.

Suitable pH adjusting additives include HCl or NaOH in amounts sufficient to adjust the pH of a topical pharmaceutical composition.

### 4. Methods of Treatment

Embodiments of the present disclosure include methods of treating cancer in a subject in need thereof, comprising administering to the subject a therapeutically effective amount of a compound described herein (e.g., a compound of formula (I), or a pharmaceutically acceptable salt thereof).

In some embodiments, the disclosure provides a method of treating prostate cancer in a subject in need thereof, comprising administering to the subject a therapeutically effective amount of a compound described herein (e.g., a compound of formula (I) or a pharmaceutically acceptable salt thereof), or a pharmaceutical composition described herein (e.g., a pharmaceutical composition comprising a compound of formula (I) or a pharmaceutically acceptable salt thereof). In some embodiments, the prostate cancer is hormone-dependent prostate cancer. In some embodiments, the prostate cancer is hormone-independent prostate cancer. In some embodiments, the prostate cancer is castration-resistant prostate cancer. In some embodiments, the cancer is metastatic castrate-resistant prostate cancer.

In the methods described herein, a compound or pharmaceutical composition may be administered to the subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; or by implant of a depot, for example, subcutaneously or intramuscularly. Additional modes of administration may include adding the compound and/or a composition comprising the compound to a food or beverage, including a water supply for an animal, to supply the compound as part of the animal’s diet.

It will be appreciated that appropriate dosages of the compounds, and compositions comprising the compounds, can vary from patient to patient. Determining the optimal dosage will generally involve the balancing of the level of therapeutic benefit against any risk or deleterious side effects of the treatments of the present disclosure. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g_{.}, in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician. In general, a suitable dose of the compound is in the range of about 100 µg to about 250 mg per kilogram body weight of the subject per day.

The compound or composition may be administered once, on a continuous basis (e.g. by an intravenous drip), or on a periodic/intermittent basis, including about once per hour, about once per two hours, about once per four hours, about once per eight hours, about once per twelve hours, about once per day, about once per two days, about once per three days, about twice per week, about once per week, and about once per month. The composition may be administered until a desired reduction of symptoms is achieved.

A compound described herein may be used in combination with other known therapies. Administered “in combination,” as used herein, means that two (or more) different treatments are delivered to the subject during the course of the subject’s affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

A compound or composition described herein and the at least one additional therapeutic agent can be administered simultaneously, in the same or in separate compositions, or sequentially. For sequential administration, the compound described herein can be administered first, and the additional agent can be administered subsequently, or the order of administration can be reversed.

In some embodiments, a compound described herein is administered in combination with other therapeutic treatment modalities, including surgery, radiation, cryotherapy, cryosurgery, and/or thermotherapy. Such combination therapies may advantageously utilize lower dosages of the administered agent and/or other chemotherapeutic agent, thus avoiding possible toxicities or complications associated with the various therapies. The phrase “radiation” includes, but is not limited to, external-beam therapy which involves three dimensional, conformal radiation therapy where the field of radiation is designed to conform to the volume of tissue treated; interstitial-radiation therapy where seeds of radioactive compounds are implanted using ultrasound guidance; and a combination of external-beam therapy and interstitial-radiation therapy.

In some embodiments, the compound described herein is administered with at least one additional therapeutic agent, such as a chemotherapeutic agent. In certain embodiments, the compound described herein is administered in combination with one or more additional chemotherapeutic agents. The chemotherapeutic agent may be a chemotherapeutic agent identified on the “A to Z List of Cancer Drugs” published by the National Cancer Institute. In some embodiments, the chemotherapeutic agent is selected from abiraterone, apalutamide, bicalutamide, cabazitaxel, capecitabine, cyclophosphamide, darolutamide, degarelix, docetaxel, dutasteride, enzalutamide, estradiol, estramustine, finasteride, flutamide, goserelin, histrelin, leuprolide, mitoxantrone, nilutamide, olaparib, radium-223, rucaparib, sipuleucel-T, and triptorelin.

### 5. Examples

It will be readily apparent to those skilled in the art that other suitable modifications and adaptations of the methods of the present disclosure described herein are readily applicable and appreciable and may be made using suitable equivalents without departing from the scope of the present disclosure or the aspects and embodiments disclosed herein. The disclosure will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

### Example 1

### FAP Expression in Multiple Tumor-Infiltrating Populations

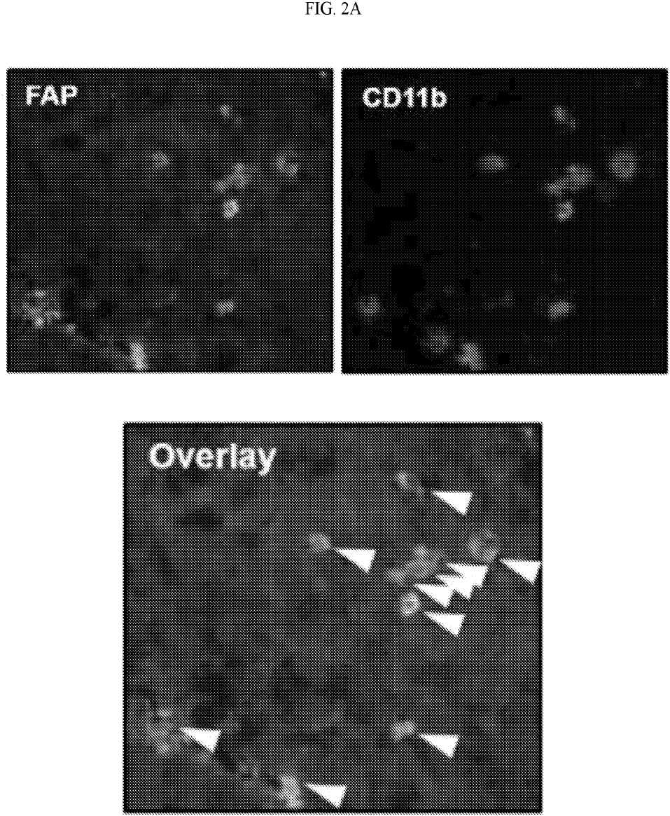

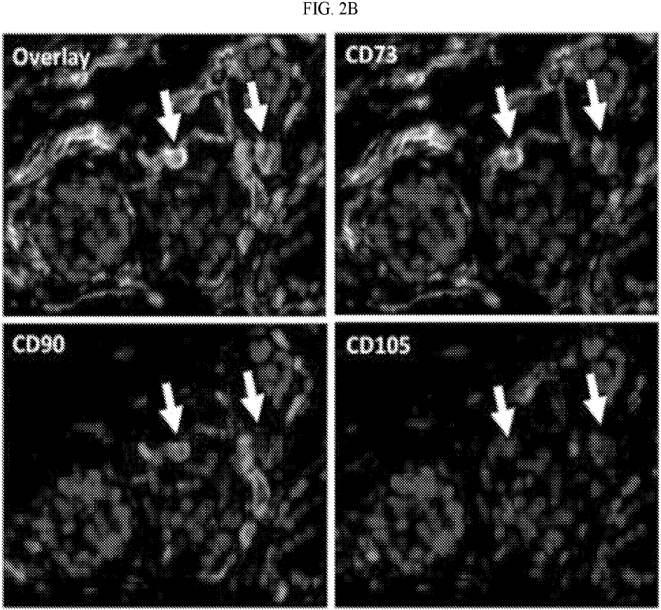

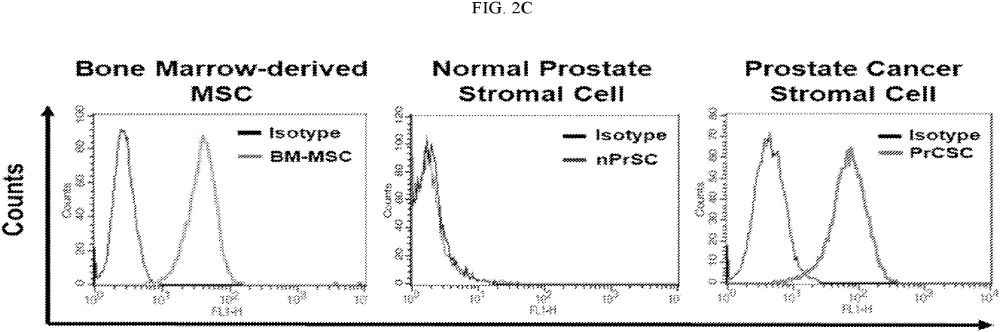

A consequence of the chronic inflammation in prostate tissue is the recruitment and polarization of multiple mediators of immune function (Brennen et al. Endocr Relat Cancer 20, R269-290 (2013); De Marzo et al. Nat Rev Cancer 7, 256-269 (2007); Sfanos et al. Histopathology 60, 199-215 (2012); De Marzo et al. Am J Pathol 155, 1985-1992 (1999)). A significant number of infiltrating FAP+ cells in human prostate cancer (PCa) are CD11b+ myeloid cells (FIG. 2A).

In extensive systematic analyses of benign and malignant prostate tissue obtained from patient tumor samples(n >50) across the spectrum of disease and patient characteristics, MSCs have been shown to be among the crucial FAP+ populations recruited to PCa foci. Using multiple orthogonal methods, including flow cytometry, in situ hybridization, radiolabeling in vitro and in vivo functional assays, in addition to a novel triple-label immunofluorescence assay developed based on canonical MSC markers (FIG. 2B), FAP+ MSCs have been documented as a key component of the PCa-associated stroma and their recruitment occurs throughout disease progression; a fact further demonstrated by the detection of MSCs in mCRPC lesions from a rapid autopsy program (Brennen et al. Oncotarget 8, 46710-46727 (2017)). MSCs are essential to tissue repair and recruited to sites of tissue damage in response to inflammatory stimuli, such as CXCL12, CCL2, CCL5, IGF-1, and TGF-β; all upregulated in PCa (Brennen et al. Endocr Relat Cancer 20, R269-290 (2013); Spaeth et al. Gene Ther 15, 730-738 (2008); Wan et al. Stem Cells 30, 2498-2511 (2012); Li et al. Biochem Biophys Res Commun 356, 780-784 (2007); Ponte et al. Stem Cells 25, 1737-1745 (2007)). Tumor-infiltrating MSCs differentiate into CAFs, which also have significant pro-tumorigenic properties (Brennen et al. Prostate 76, 552-564 (2016); Mishra et al. Cancer Res 68, 4331-4339 (2008); Paunescu et al. J Cell Mol Med 15, 635-646 (2011); Madar et al. Trends Mol Med 19, 447-453 (2013); Borriello et al. Cancer Res 77, 5142-5157 (2017); Barcellos-de-Souza et al. Stem Cells 34, 2536-2547 (2016); Hughes et al. Cancer Res 79, 3636-3650 (2019)). Like bone marrow-derived MSCs (Bae et al. Br J Haematol 142, 827-830 (2008)), PCa-derived MSCs and CAFs (i.e. PrCSCs) express FAP, but stromal cells in normal prostate tissue (i.e. nPrSCs) do not (FIG. 2C); consistent with the lack of FAP expression in other normal tissues.

### Example 2

### Compound Syntheses

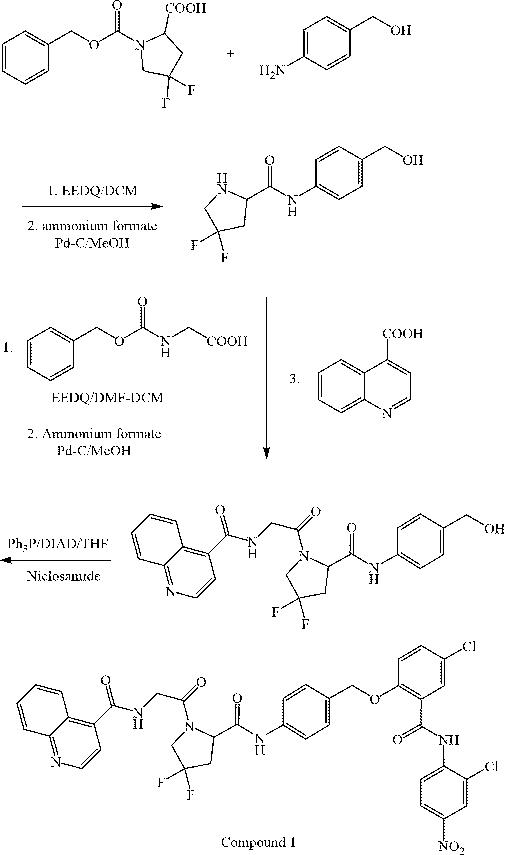

Compound 1. N-Cbz-4,4-difluoro-L-Proline was reacted with paraaminobenzyl alcohol using N-Ethoxycarbonyl-2-ethoxy-1,2-dihydroquinoline (EEDQ) as coupling reagent in dichloromethane (DCM). Upon purification and analysis, the Cbz protecting group was removed by reacting the product with a mixture of 10% palladium on carbon and ammonium formate in methanol. Following purification and analysis, the product obtained was coupled to N-Cbz-Glycine using EEDQ in DMF-DCM mixture (1:1). This was purified, analyzed and Cbz was removed as previously done. The deprotected product was purified, analyzed and then reacted with quinoline-4-carboxylic acid using EEDQ as coupling reagent in DMF-DCM (1:1) mixture followed by purification and analysis. Mitsunobu reaction was carried out between this product and niclosamide using triphenylphosphine (Ph₃P) and diisopropyl azodicarboxylate (DIAD) in tetrahydrofuran (THF). The final product was purified and analyzed to confirm that it is the desired FAP activated prodrug of niclosamide by NMR, MS and HPLC analysis. MS analysis gave an m/z value of 777.3730.

The general synthesis scheme can be used to readily couple other cytotoxic agents containing either hydroxyl, amine or carboxyl functional groups to the FAP substrate using an appropriate self-cleaving linker. Additional compounds are shown in Table 1.

TABLE 1

Exemplary compounds and data

| Compound No. | Structure | MS data |
|---|---|---|
| 2 | 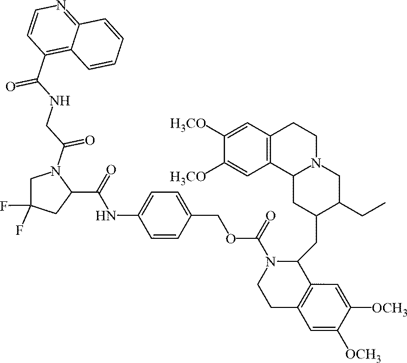 [SMILES: CCC1CN2CCc3cc(OC)c(OC)cc3C2CC1CC1c2cc(OC)c(OC)cc2CCN1C(=O)OCc1ccc(NC(=O)C2CC(F)(F)CN2C(=O)CNC(=O)c2ccnc3ccccc23)cc1] | 975.1328 |
| 3 | 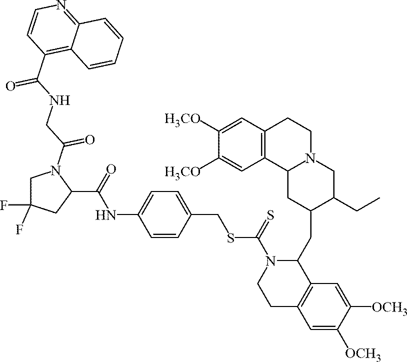 [SMILES: CCC1CN2CCc3cc(OC)c(OC)cc3C2CC1CC1c2cc(OC)c(OC)cc2CCN1C(=S)SCc1ccc(NC(=O)C2CC(F)(F)CN2C(=O)CNC(=O)c2ccnc3ccccc23)cc1] | 1007.1248 |
| 4 | 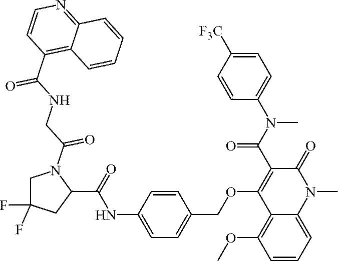 [SMILES: COc1cccc2c1c(OCc1ccc(NC(=O)C3CC(F)(F)CN3C(=O)CNC(=O)c3ccnc4ccccc34)cc1)c(C(=O)N(C)c1ccc(C(F)(F)F)cc1)c(=O)n2C] | 856.7570 |
| 5 | 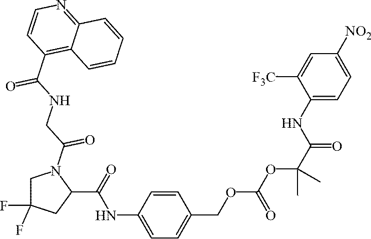 [SMILES: CC(C)(OC(=O)OCc1ccc(NC(=O)C2CC(F)(F)CN2C(=O)CNC(=O)c2ccnc3ccccc23)cc1)C(=O)Nc1ccc([N+](=O)[O-])cc1C(F)(F)F] | 786.5480 |
| 6 | 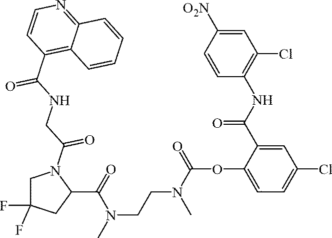 [SMILES: CN(CCN(C)C(=O)C1CC(F)(F)CN1C(=O)CNC(=O)c1ccnc2ccccc12)C(=O)Oc1ccc(Cl)cc1C(=O)Nc1ccc([N+](=O)[O-])cc1Cl] | 786.4610 |
| 7 | 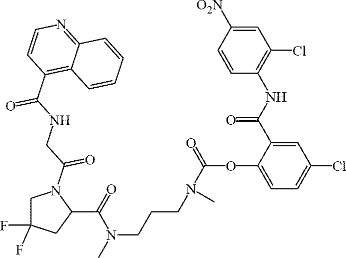 [SMILES: CN(CCCN(C)C(=O)C1CC(F)(F)CN1C(=O)CNC(=O)c1ccnc2ccccc12)C(=O)Oc1ccc(Cl)cc1C(=O)Nc1ccc([N+](=O)[O-])cc1Cl] | 800.4830 |
| 8 | 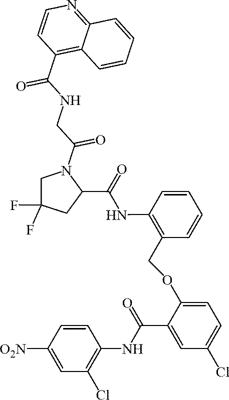 [SMILES: O=C(Nc1ccc([N+](=O)[O-])cc1Cl)c1cc(Cl)ccc1OCc1ccccc1NC(=O)C1CC(F)(F)CN1C(=O)CNC(=O)c1ccnc2ccccc12] | 777.2618 |
| 9 | 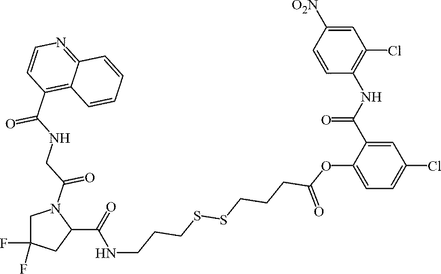 [SMILES: O=C(CCCSSCCCNC(=O)C1CC(F)(F)CN1C(=O)CNC(=O)c1ccnc2ccccc12)Oc1ccc(Cl)cc1C(=O)Nc1ccc([N+](=O)[O-])cc1Cl] | 863.1131 |
| 10 | 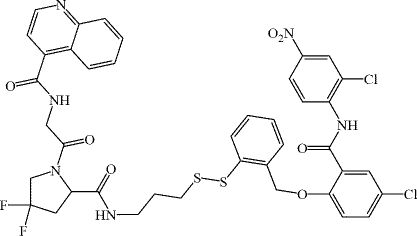 [SMILES: O=C(Nc1ccc([N+](=O)[O-])cc1Cl)c1cc(Cl)ccc1OCc1ccccc1SSCCCNC(=O)C1CC(F)(F)CN1C(=O)CNC(=O)c1ccnc2ccccc12] | 882.1132 |

### Example 3

### Efficacy Data

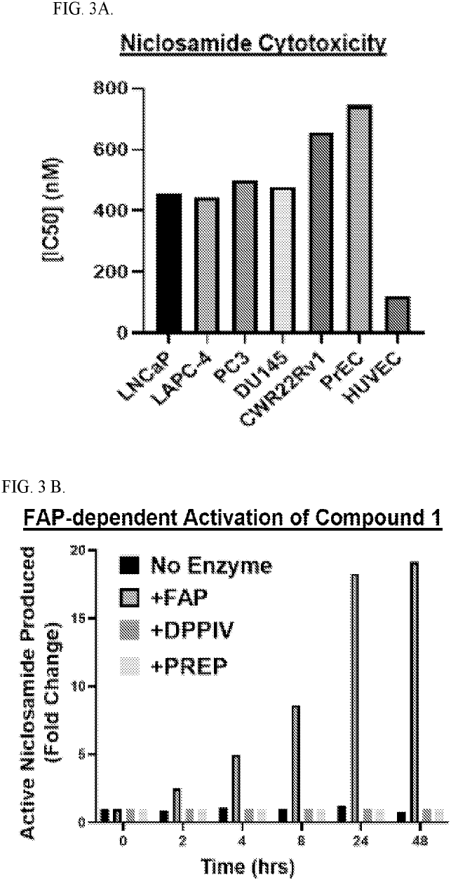

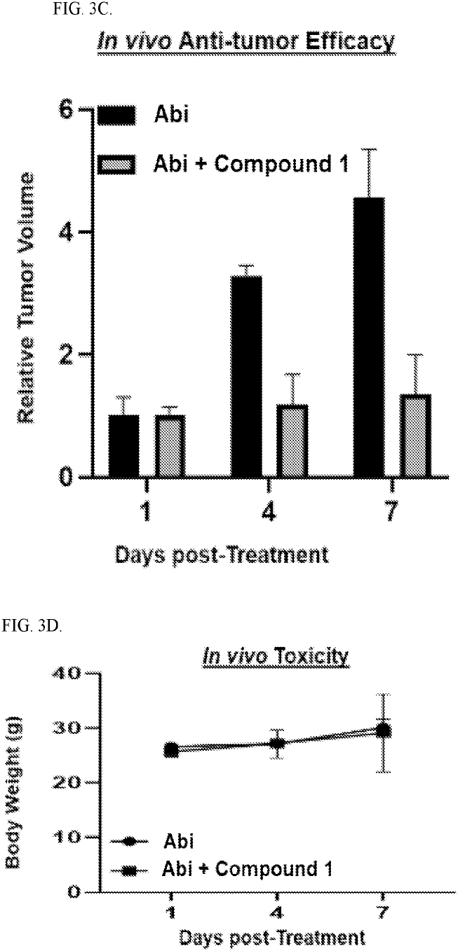

Based on its non-selective mechanism of action, niclosamide has nanomolar potency against all cells tested [Avg IC50: <500 nM, (FIG. 3A)].

An optimized LC/MS/MS protocol has been developed to accurately quantify niclosamide and niclosamide prodrugs in biological matrices for biochemical and pharmacokinetic analyses. Column: Luna 2.5u C18, 100 mm ×2.5 mm (Phenomenex 00D-4446-B0). Mobile phase: A = 0.5% acetonitrile/H₂O/0.1% formic acid; B= 95% acetonitrile/H₂O/0.1% formic acid; Flow = 150 µL/min.

| Time | A% | B% |
|---|---|---|
| 0 | 95 | 5 |
| 2 | 95 | 5 |
| 6 | 2 | 98 |
| 10 | 2 | 98 |
| 12 | 95 | 5 |
| 15 | 95 | 5 |

The MS conditions for niclosamide are Negative mode with the following voltage settings:

| | | |
|---|---|---|
| Q1 M/Z | -326.8 | -326.8 |
| Q3 M/Z | -173 | -171 |
| Declustering Pot. | -25 | -25 |
| Entrance Pot. | -10 | -10 |
| Coll. Energy | -38 | -36 |
| Coll. Cell Exit Pot. | -15 | -7 |

The MS conditions for NF1 are in Positive mode with the following voltage settings:

| | | | |
|---|---|---|---|
| Q1 M/Z | 777 | 777 | 777 |
| Q3 M/Z | 451 | 106 | 185 |
| Declustering Pot. | 131 | 131 | 131 |
| Entrance Pot. | 10 | 10 | 10 |
| Coll. Energy | 25 | 43 | 75 |
| Coll. Cell Exit Pot. | 22 | 10 | 14 |

Niclosamide has a retention time of approximately 10.1 minute and Compound 1 elutes approximately at 11.0 minute.

Using this validated assay, FAP-specific activation of Compound 1 was observed, with an ~20-fold increase in active niclosamide over 24 hrs with no production observed with DPPIV, PREP, or in the absence of enzyme (FIG. 3B). Importantly, niclosamide is non-selective once activated and toxic to all cells. Therefore, a significant bystander effect killing prostate cancer cells is also expected once the toxin is activated in the TME. Importantly, due to its high lipophilicity, niclosamide will rapidly partition into nearby cells and not “leak” out of the local TME once “activated,” thereby sparing toxicity to peripheral normal tissues.

Animals were treated with either abiraterone at 10 mg/kg by daily oral gavage alone or together with Compound 1 at 50 mg/kg IV on d0 and d4. Toxicity was monitored by measuring body weight and body score (i.e., appearance, lethargy, etc.). Treatment with Compound 1 produced significant anti-tumor efficacy in vivo against abiraterone-resistant CRPC tumors [FIG. 3C, Myc-CaP-CR] with no evidence of toxicity (FIG. 3D).

### Example 4

### Characterization Methods

In vitro Characterization of Enzymatic Activation Kinetics and FAP-specific Cellular UptakelCytotoxicity: Kinetics of compound activation by FAP is performed as previously described (Aggarwal et al. Biochemistry 47, 1076-1086 (2008); Denmeade et al. Sci TranslMed 4, 140ra186 (2012)). Briefly, increasing concentrations of the test compound (0-10 µM) are incubated with rhFAP, rhDPPIV, or rhPREP [20 nM] (R&D Systems) at 37° C. while shaking. Aliquots are taken to determine concentrations of the test compound and active drug compound at time points ranging from 0-24 hrs using the LCMS method described above in Example 4. Kinetic constants (Km and kcat) are calculated on the basis of the rate of hydrolysis (v) during the linear phase using Michaelis-Menten plots (v vs [S]) with nonlinear regression analysis. Cellular uptake in the presence or absence of rhFAP is determined in cell lysates using the same methodology. Cytotoxicity is determined via MTT in the presence or absence of rhFAP and/or a FAP-specific inhibitor (Jansen et al. J Med Chem 57, 3053-3074 (2014).

Maximum-tolerated Dose (MTD) in vivo: Oral maximum-tolerated dose (MTD) of the protoxin compared to the drug compound itself is determined as previously described (Brennen et al. J Natl Cancer Inst 104, 1320-1334 (2012)) (n = 5/dose in escalating dose levels starting at 500 mg/kg/day by oral gavage). Animals are monitored daily for signs of distress [i.e. weight loss (>20%), anorexia, lethargy, labored respiration, hunched posture, uncoordinated movements, cyanosis, vocalization, or crying, etc.]; if observed, animals are euthanized immediately according to IACUC-approved protocols.

in vivo Efficacy and Toxicity: For efficacy studies in the PDX models, male NSG mice inoculated with BCaP-1, LvCaP-1, LvCaP-2, or SkCaP-1 in intact hosts or their enzalutamide-resistant variants in surgically-castrated hosts are treated at the MTD via oral gavage (n = 15/grp) and compared to vehicle controls. Tumor volumes and body weight recorded 2x/wk as we have previously described103,109. At 1 wk post-treatment and end of study, 3 animals/grp sacrificed to harvest tumors, tissues, and peripheral blood (PB). Tumors are portioned for histology, biodistribution, and analysis of stromagenesis. Every 2 wks, 50 µl of blood collected via retro-orbital sampling analyzed using a Hemavet 950FS instrument for CBC to assess hematopoietic function.

Pharmacokinetics (PK): Niclosamide concentrations in plasma and tumor samples are quantified via LCMS (Schweizer et al. PLoS One 13, e0198389 (2018)). Briefly, heparinized plasma collected at 0, 0.5, 1, 2, 4, 8, and 24 hrs following the first dose are mixed with an internal standard. Tumor/tissues collected at 0, 8, 24, 48, 72, and 96 hrs post-steady state [i.e. 1 wk daily dosing, (n = 3/time pt)]. Tissue supernatants and plasma samples are injected onto a reverse-phase C18 column (Phenomenex Luna) on a Sciex 6500 triple quad liquid chromatography mass spectrometry (LCMS) system with the MS operated in ESI-negative mode to quantify the following transition pair for niclosamide (324.8/170.9 m/z) using a 10 pt calibration curve.

Stromagenesis: To determine immune-independent effects of the compounds, multiple assays evaluating stromagenesis are performed (n = 3 tumors/grp). Masson’s TriChrome staining is performed to assess collagen and smooth muscle content. Intra-tumoral collagen is assessed by picosirius red staining (i.e. measure of collagen dis/organization) and quantification of total collagen content by hydroxyproline assay (Santos et al. J Clin Invest 119, 3613-3625 (2009); Jackson et al. Methods Biochem Anal 15, 25-76 (1967)). For picosirius staining, slides are incubated in a picric acid-saturated solution are washed and visualized using a Zeiss Axioplan II with polarization capabilities available in the SKCCC Imaging Core. Hydroxyproline quantified by digesting tumors sequentially in 6N HCI, followed by neutralization, mixing with chloramine T solution, then Ehrlich’s solution. Abs is be measured at 570 nm, and compared to a standard cis-4-hydroxy-L-proline curve (Jackson 1967). Angiogenesis is evaluated by quantifying CD34+ microvessel density.

### Example 5

### In Vivo Data for Compound 4

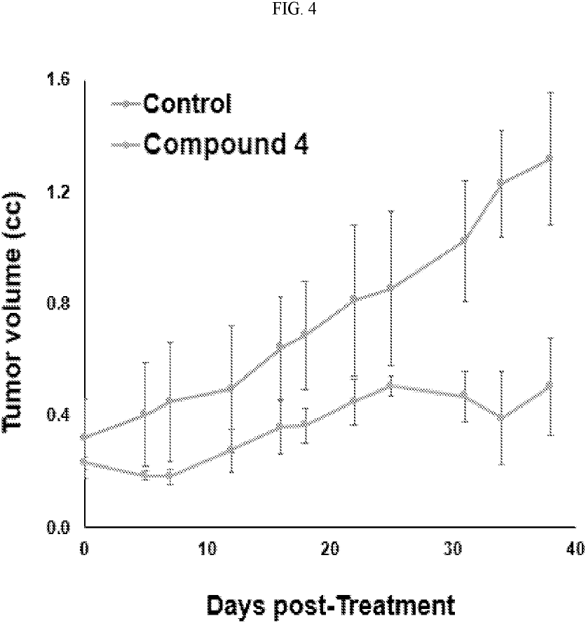

Intact male NSG mice (3-4 per group) bearing SkCaP-1 (human PCa xenograft) were treated with 30 mg/kg Compound 4 (a tasquinimod compound) i.p. daily, or a control (DMSO). Tumor volume was measured and results are shown in FIG. 4. Compound 4 decreases tumor growth compared to the control.

### Example 6

### Kinetics of FAP-Dependent Activation of Compound 1 In Vitro

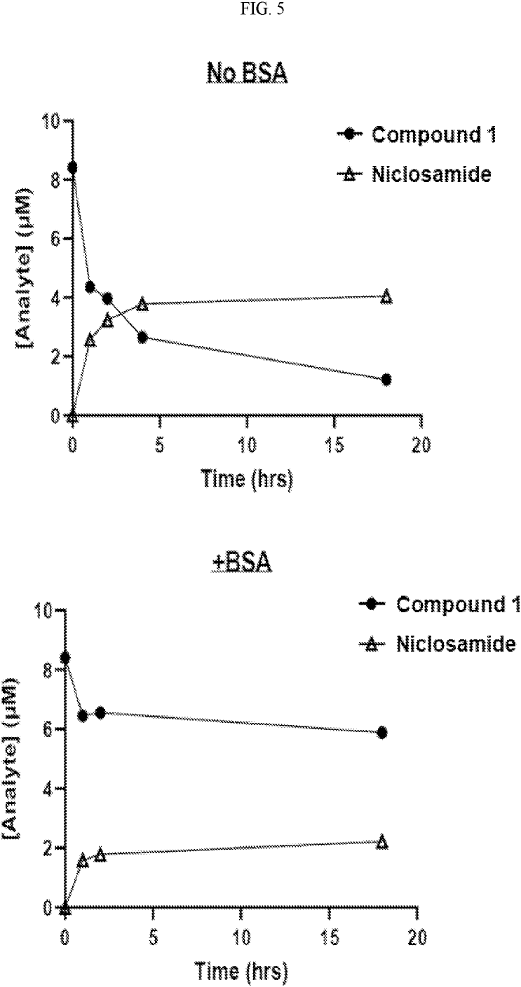

Compound 1 was incubated in Buffer (50 mM Tris-HCl, pH 7.4; 150 mM NaCI) +/-0.4% BSA in glass vials, mixing at 37° C. Aliquots were taken at indicated time points and the presence of Compound 1 (prodrug) and Niclosamide (active compound) were quantified via HPLC. Results are shown in FIG. 5, and show time-dependent increase in the active compound and concomitant decrease in the prodrug over time. BSA inhibits production of the active compound, likely due to protein binding of the prodrug that sterically hinders access of the activating enzyme (i.e., FAP).

### Example 7

### Seahorse Mitochondrial Stress Test Assay for Compound 1

A mitochondrial stress test assay was performed using the Agilent Seahorse XF Cell Mito Stress Test Kit (catalog no. 103015-100), a widely-used assay for assessing mitochondrial function. Compound 1 or niclosamide (1 µM) was incubated overnight at 37° C. shaking, with or without FAP (20 nM) in Seahorse Media. This mixture was added to LNCaP cells (60 k cells) the next day, immediately prior to starting the assay (0.5 µM final concentration).

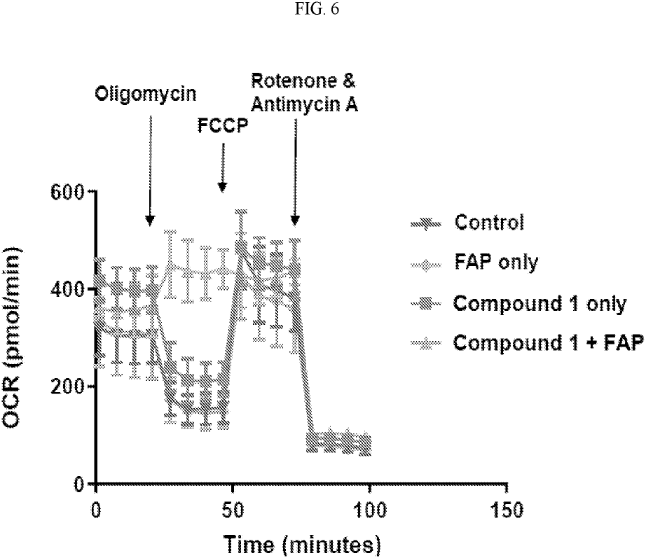

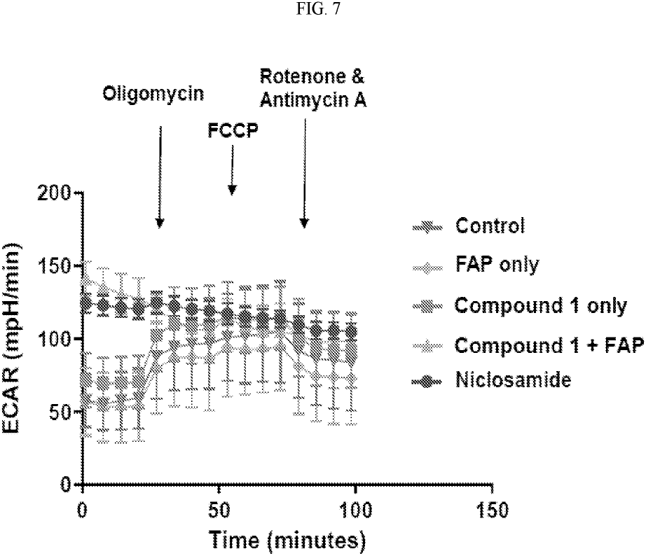

Results for are shown in FIGS. 6 and 7. Data in FIG. 6 show that Compound 1 activated by FAP, but not in its absence, it uncouples mitochondria as documented by no change in oxygen consumption rate (OCR) with the addition of Oligomycin (ATP Synthase (Complex V) inhibitor) or carbonyl cyanide 4-(trifluoromethoxy)phenylhydrazone (FCCP, a known mitochondrial uncoupler), while no change in non-mitochondrial respiration is observed as documented following rotenone and antimycin A addition (Complex I and III inhibitors, respectively). Data in FIG. 7 show that the extracellular acidification rate (ECAR) in the presence of Compound 1 activated by FAP, but not in its absence, does not change with the addition of oligomycin, indicating that cells are already relying upon glycolysis due to non-functional mitochondrial oxidative phosphorylation. Furthermore, baseline ECAR is already at maximum, indicating complete dependence on glycolysis.

### Example 8

### Efficacy of Compound 1 Against Human Castration-Resistant Prostate Cancer (CWR22-H) In Vivo

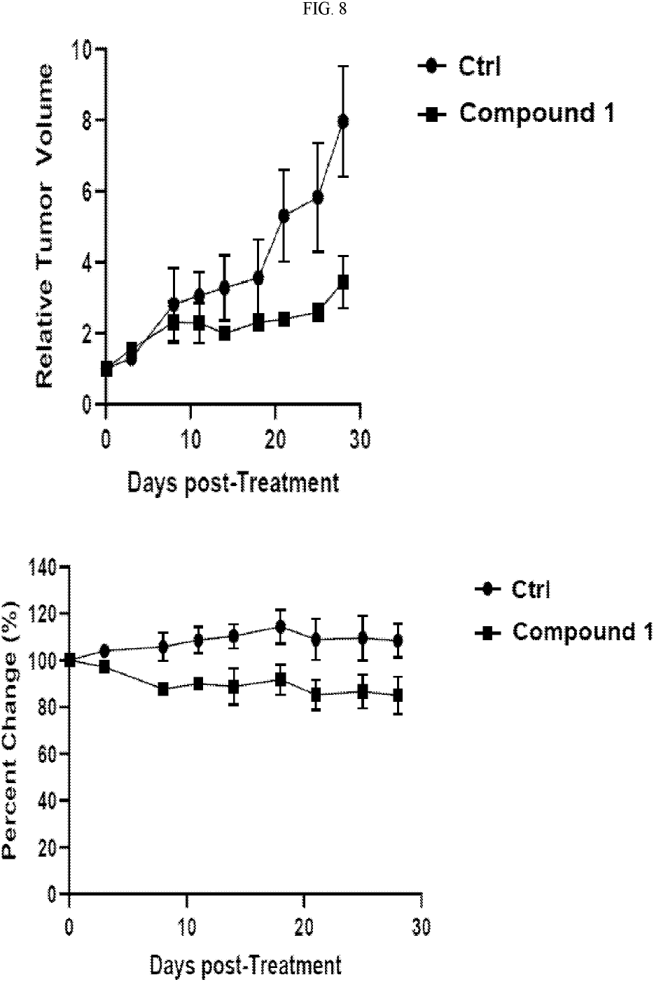

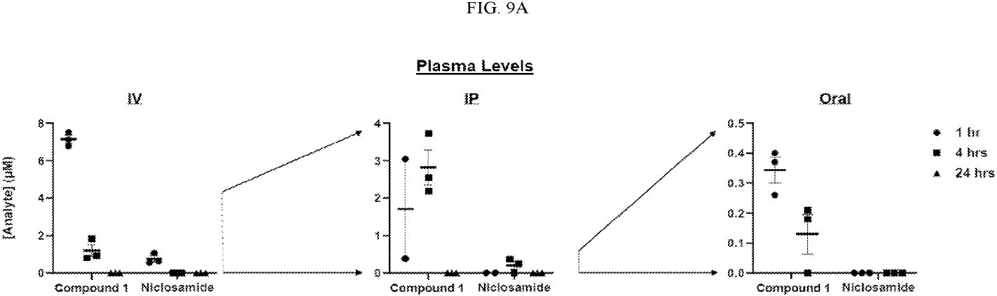

Castrated male NSG mice were inoculated with castration-resistant CWR22-H tumors in 50% Matrigel. When tumors reached ~0.2 cc, treatment with Compound 1 (40 mg/kg) via intraperitoneal (I.P.) injection was initiated. Tumor volumes and body weights were monitored 2x/wk. Data are shown in FIG. 8. Anti-tumor activity was observed against castration-resistant CWR22-H tumors with daily IP dosing of Compound 1 (40 mg/kg). Treatment was well-tolerated as documented by weight loss of <20%.

### Example 9

### Pharmacokinetic Data for Compound 1

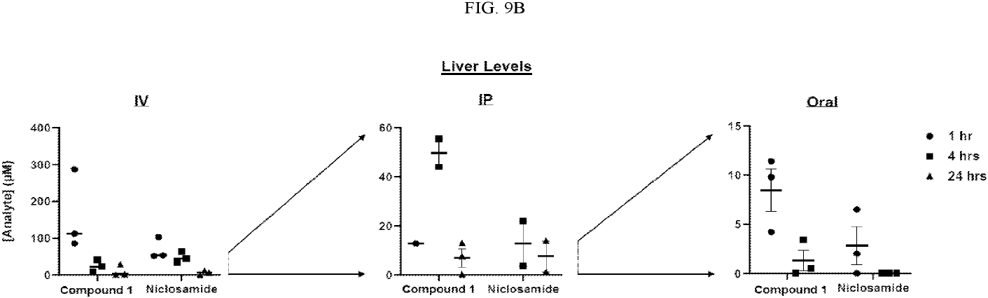

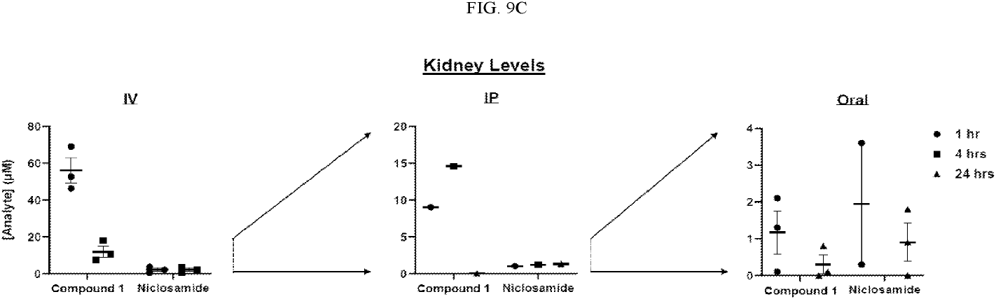

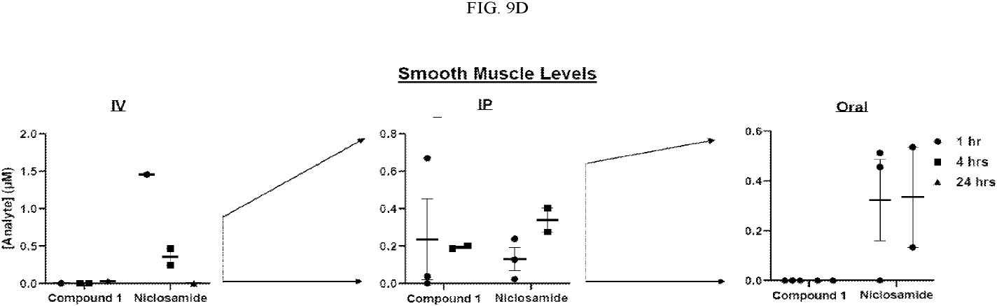

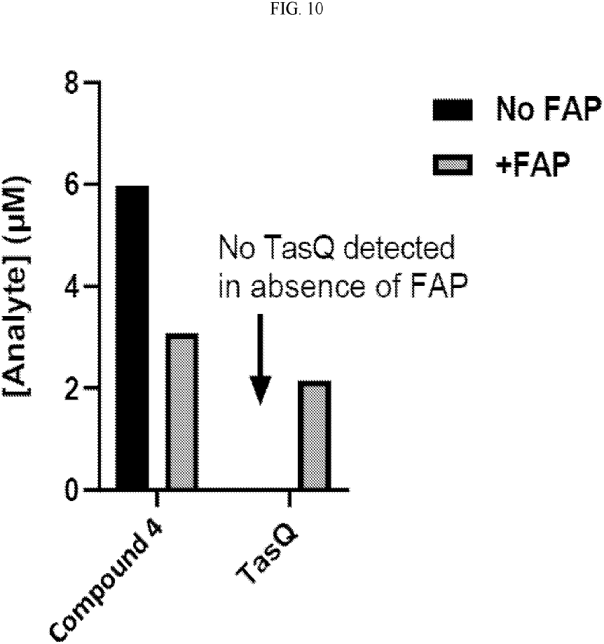

Plasma, liver, kidney, and smooth muscle levels of Compound 1 and niclosamide were determined following dosing with Compound 1 (40 mg/kg) either intravenously, intraperitoneally, or orally. Results are shown in FIGS. 9A-9D.

As shown in FIG. 9A, low micromolar (~7 µM) levels at Cmax are achieved in the plasma at 1 hr post-IV dosing. Half-life is on the order of a 3-4 hrs. If the same dose is delivered IP, ~50% of the IV dose are achieved in the plasma with a delayed Cmax at ~4 hrs due to absorption. ~5-10% of is bioavailable if delivered orally, reaching a Cmax of ~0.4 µM at 1 hr post-dosing. <1 µM niclosamide of Compound 1 dose (i.e. <10%) is present in the plasma independent route of administration.

As shown in FIG. 9B, ~100 µM levels (i.e. ~10-15x plasma concentrations) are achieved in the liver at 1 hr post-IV dosing. If the same dose is delivered IP, ~50% of the IV dose are achieved in the liver at ~4 hrs due to absorption. ~10% of is bioavailable if delivered orally, reaching ~10 µM at 1 hr post-dosing, higher concentrations than detected in the plasma are expected due to first pass kinetics. Niclosamide is present at ~25-50% of Compound 1 concentrations, higher conversion presumably due to liver metabolism.

As shown in FIG. 9C, ~50 µM are achieved in the kidney at 1 hr post-IV dosing, higher levels likely due to excretion. If the same dose is delivered IP, ~30% of the IV dose are achieved in the kidney at ~4 hrs due to absorption. If delivered orally, ~1 µM Compound lis achieved in the kidney at 1 hr post-dosing and equivalent levels of niclosamide are present at 1 and 4 hrs. <10% converted to niclosamide if Compound 1 is administered IV or IP.

As shown in FIG. 9D, very low levels (<1 µM) of Compound 1are detected in smooth muscle at any time point tested independent of route of administration. Niclosamide is detected in SM, but also typically <1 µM (i.e. <5-10% of administered dose based on plasma concentrations, <1% based on liver concentrations) indicating good stability of the prodrug as a systemic therapy.

### Example 10

### FAP-Dependent Activation Data for Compound 4 In Vitro

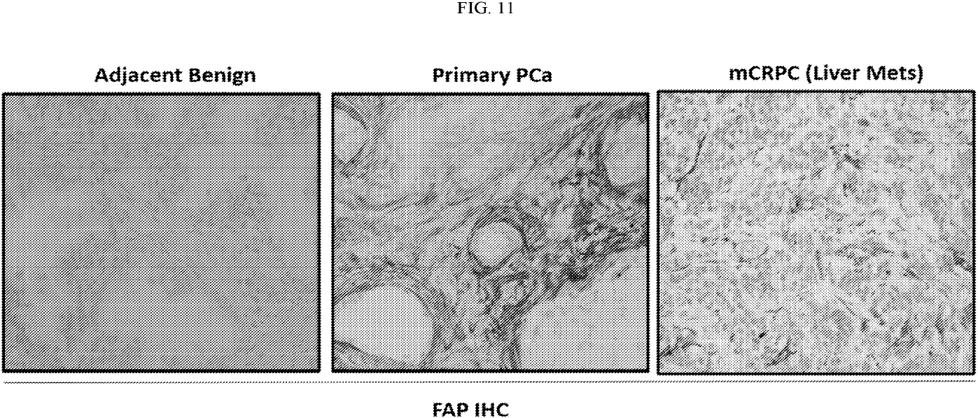

Compound 4 was incubated in buffer +/- rhFAP (20 nM) overnight (~16 hrs), mixing at 37° C. Aliquots taken at indicated time points and the presence of Compound 4 (prodrug) and Tasquinimod (TasQ) were quantified via HPLC. Data are shown in FIG. 10, and confirm FAP-dependent activation of Compound 4 to produce the active compound, tasquinimod.

### Example 11

### Immunohistochemistry

IHC Protocol: anti-FAP, Clone SP325 (#ab227703; human-specific): Immunolabeling for FAPa was performed on FFPE sections on a Ventana Discovery Ultra autostainer (Roche Diagnostics). Briefly, following dewaxing and rehydration on board, epitope retrieval was performed using Ventana Ultra CC1 buffer (catalog# 6414575001, Roche Diagnostics) at 96° C. for 64 minutes. Primary antibody, anti- FAPa (1:100 dilution; catalog# ab227703, lot# GR3208068-1 Abcam) diluted in Ventana antibody diluent with casein (catalog# 760-219, Roche Diagnostics) was applied at 36° C. for 60 minutes. Primary antibodies were detected using an anti-rabbit HQ detection system (catalog# 7017936001 and 7017812001, Roche Diagnostics), amplified by Discovery AMP Multimer (catalog # 6442544001, Roche Diagnostics) followed by Chromomap DAB IHC detection kit (catalog # 5266645001, Roche Diagnostics), counterstaining with Mayer’s hematoxylin, dehydration and mounting. Images are shown in FIG. 11, which show overexpression of FAP in primary and metastatic human prostate cancer compared to adjacent benign prostate tissue.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

1. A compound of formula (I): 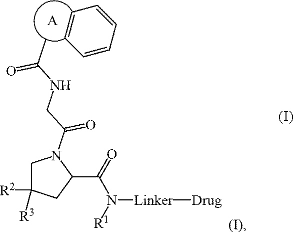
or a pharmaceutically acceptable salt thereof, wherein:
R¹ is selected from hydrogen and methyl;
R² and R³ are each independently halogen; and
A is a 5- or 6-membered heteroaryl or heterocyclic ring.

2. The compound of claim 1, or a pharmaceutically acceptable salt thereof, wherein R¹ is hydrogen.

3. The compound of claim 1 or claim 2, or a pharmaceutically acceptable salt thereof, wherein R² and R³ are each fluoro.

4. The compound of any one of claims 1-3, or a pharmaceutically acceptable salt thereof, wherein A is a 5- or 6-membered heteroaryl having one heteroatom selected from N, O, and S.

5. The compound of any one of claims 1-3, or a pharmaceutically acceptable salt thereof, wherein A is selected from thiophene, furan, and pyridine.

6. The compound of any one of claims 1-5, wherein the compound has formula (Ia): 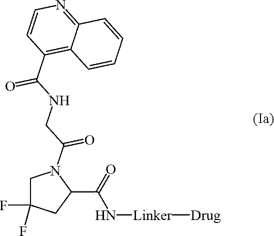
.

7. The compound of any one of claims 1-6, or a pharmaceutically acceptable salt thereof, wherein the linker is a self-cleaving linker.

8. The compound of any one of claims 1-6, or a pharmaceutically acceptable salt thereof, wherein the linker has a formula selected from: 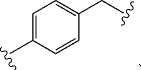 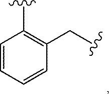 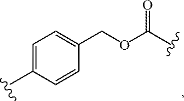 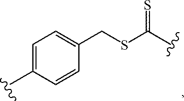 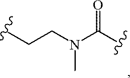 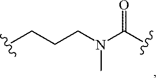 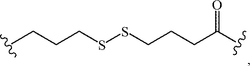 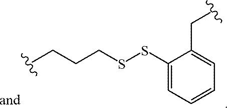
.

9. The compound of any one of claims 1-8, or a pharmaceutically acceptable salt thereof, wherein the drug is selected from niclosamide, emetine, 2-hydroxyflutamide, and tasquinimod.

10. The compound of claim 1, wherein the compound is selected from: 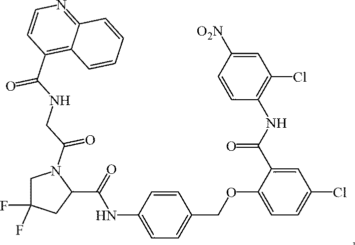 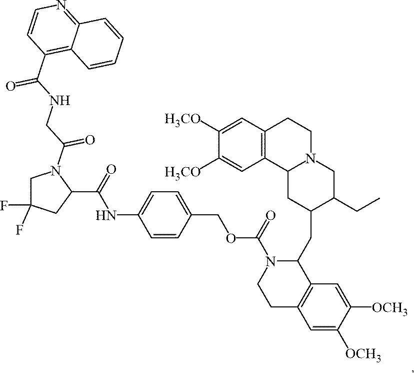 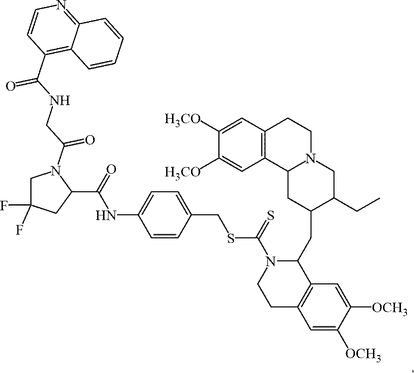 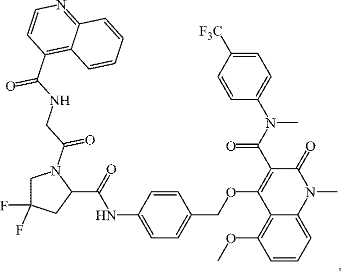 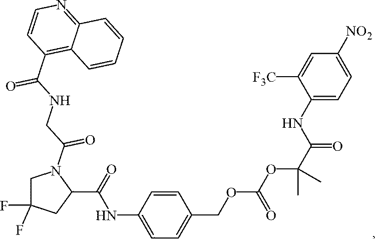 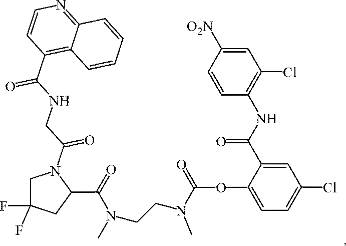 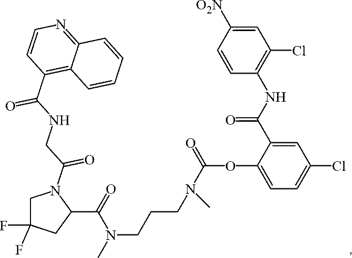 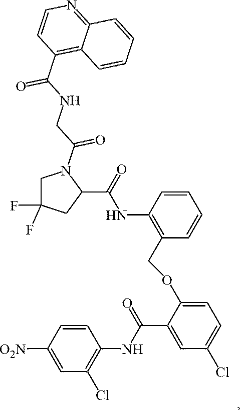 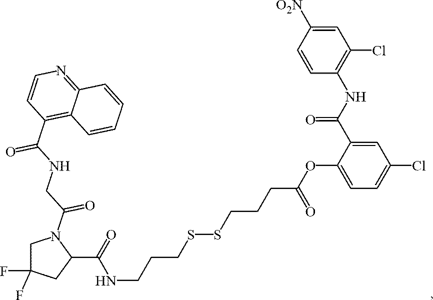 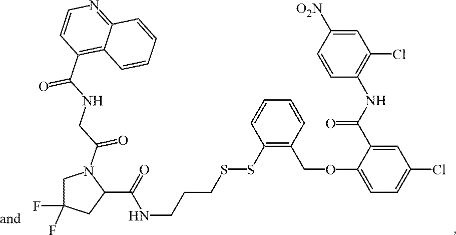
and a pharmaceutically acceptable salt thereof.

11. A pharmaceutical composition comprising a compound of any one of claims 1-10 and a pharmaceutically acceptable carrier.

12. A method of treating cancer in a subject in need thereof, comprising administering to the subject a therapeutically effective amount of a compound of any one of claims 1-10.

13. The method of claim 12, wherein the cancer is prostate cancer.

14. The method of claim 12, wherein the prostate cancer is metastatic castration-resistant prostate cancer.

15. The method of any one of claims 12-14, further comprising administering an additional chemotherapeutic agent to the subject.

16. The method of any one of claims 12-15, wherein the subject is a human.

17. Use of a compound of any one of claims 1-10, or a pharmaceutical composition of claim 11, in the treatment of cancer.

18. The use of claim 17, wherein the cancer is prostate cancer.

19. The use of claim 18, wherein the prostate cancer is metastatic castration-resistant prostate cancer.
